(19) United States
(12) Patent Application Publication
(10) Pub. No.: US 2025/0302773 A1
(43) Pub. Date: Oct. 2, 2025

(54) COMPOSITIONS COMPRISING MEAI AND N-ACYLETHANOLAMINES AND USES THEREOF

(71) Applicant: CLEARMIND MEDICINE INC., Tel Aviv (IL)

(72) Inventor: Adi ZULOFF-SHANI, Tel Aviv (IL)

(73) Assignee: CLEARMIND MEDICINE INC., Vancouver, BC (CA)

(21) Appl. No.: 18/844,341

(22) PCT Filed: Mar. 6, 2023
(86) PCT No.: PCT/IB2023/052091

§ 371 Date: Sep. 6, 2024

Related U.S. Application Data
(60) Provisional application No. 63/317,257, filed on Mar. 7, 2022.

(51) Int. Cl. A61K 31/135; A61K 31/164; A61P 25/32

(52) U.S. Cl. CPC A61K 31/135; A61K 31/164; A61P 25/32

(57) ABSTRACT

The present disclosure relates to pharmaceutical compositions comprising 5-methoxy-2-aminoindan, or a salt thereof, and an N-acylethanolamine, or a salt thereof, and at least one pharmaceutically acceptable carrier and/or excipient, and their use in methods of regulating binge behavior by administrating said compositions to a subject in need thereof in a therapeutically effective amount.

### CROSS-REFERENCE TO RELATED APPLICATION

This application claims priority to U.S. Provisional Application No. 63/317,257, filed on Mar. 7, 2022, the contents of which are hereby incorporated by reference.

### FIELD OF THE DISCLOSURE

The present disclosure relates to compositions and methods for potentiating therapeutic effects and/or reducing side-effects of 5-methoxy-2-aminoindan (“MEAI”). The present disclosure provides pharmaceutical compositions comprising MEAI and N-acylethanolamines, for example, palmitoylethanolamide (“PEA”) and methods for their use in a variety of indications amenable to treatment with MEAI.

### BACKGROUND OF THE DISCLOSURE

Binging or binge behavior is a non-controlled excessive behavior of indulgence in a variety of activities such as eating, drinking, drugs, sweets, shopping, sexual conduct, and the like. It is now recognized that all types of binging are ways of dealing with negative emotions that are not rational or healthy.

Binge disorders are characterized by feelings of powerlessness, secrecy, shame, and social isolation. The occasional overindulgence becomes a real problem once a subject feels a need to binge in private, or schedule binges around (or instead of) work and social obligations. Binge eating is currently the most common eating disorder in adults, compulsive buying disorder (“shopaholism”) is increasing, and binge drinking is widespread.

The causes of any type of binge behavior can fall into three categories: psychological, chemical, and sociocultural.

Most common psychological causes of binging are anxiety, stress, and depression. While often binging is simply a way to numb unhappy feelings, it can also be a symptom of an undiagnosed mental disorder. Depression, for example, can lead to low self-esteem, body dissatisfaction, poor impulse control, and difficulty in managing feelings—all of which can trigger a binge. Naturally the pain and guilt that comes in the aftermath of a binge can trigger depression, which can trigger another binge, and so on.

Alcohol is one of the favorite, commonly used, yet most dangerous psychoactive substances that may lead to binge behavior upon excessive, uncontrolled consumption. Alcohol is consumed for several reasons, which include quenching thirst, heating or cooling the drinker, for the taste and because of the association alcoholic drinks have with other aspects of life such as food and friendship. The psychological effects of alcohol contribute to some of these reasons.

Many people with alcohol dependence find that sometimes they can control their intake and have just a couple of drinks. Whereas at other times, even though they set out with the intention of only having a couple of drinks, they lose control once they have had the first drink and then take much more than they wanted to. Often this is in the form of a binge or ‘bender.”

Alcohol consumption presents a growing problem worldwide, which some believe may have already overtaken tobacco in terms of overall health and social care costs.

Excessive and/or prolonged alcohol consumption may have some undesired physiological and psychological, including short-term, effects such as gastric irritation, anxiety disorders and other excitable states, and longer-term effects such as cirrhosis, fatty liver disease, cardiomyopathy and dementia. Alcohol consumption may lead to intoxication, which, in turn, can have serious consequences such as accidents and uncontrolled violent behavior with subsequent medical complications.

Compounds derived from 2-aminoindan have been shown to selectively bind to the dopamine D3 receptor. U.S. Pat. No. 5,708,018 discloses some 2-aminoindan derivatives and hypothesizes that these 2-aminoindan derivatives may be useful in treating CNS disorders associated with dopamine D3 receptor. One such compound is 5-methoxy-2-aminoindan (“MEAI”). The chemical structure of MEAI is:

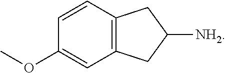

N-acylethanolamines (NAEs) are lipid-derived signaling molecules. They are formed when one of several types of acyl groups is linked to the nitrogen atom of ethanolamine. Examples of N-acylethanolamines include anandamide (the amide of arachidonic acid (20:4 omega-6) and ethanolamine), N-Palmitoylethanolamine (the amide of palmitic acid (16:0) and ethanolamine), N-Oleoylethanolamine (the amide of oleic acid (18:1) and ethanolamine), N-Stearoylethanolamine (the amide of stearic acid (18:0) and ethanolamine) and N-Docosahexaenoylethanolamine (the amide of docosahexaenoic acid (22:6) and ethanolamine).

Palmitoylethanolamide (PEA, also known as N-(2-hydroxyethyl) hexadecanamide; Hydroxyethylpalmitamide; palmidrol; N-palmitoylethanolamine; and palmitylethanolamide) is an endogenous fatty acid amide, belonging to the class of nuclear factor agonists. The chemical structure of PEA is:

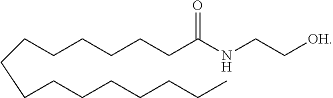 [SMILES: CCCCCCCCCCCCCCCC(=O)NCCO]

PEA has been demonstrated to bind to a receptor in the cell nucleus (a nuclear receptor) and exerts a variety of biological functions related to chronic pain and inflammation. Studies have shown that PEA interacts with distinct non-CB1/CB2 receptors, suggesting that PEA utilizes a unique “parallel” endocannabinoid signaling system. This concept was further supported by growing evidence that PEA production and inactivation can occur independently of AEA and 2-AG production and inactivation. Much of the biological effects of PEA on cells can be attributed to its affinity to PPAR (particularly PPAR-.alpha. and PPAR-.gamma.). PEA was shown to have an affinity to cannabinoid-like G-coupled receptors GPR55 and GPR119 as well as the transient receptor potential vanilloid type 1 receptor (TRPV1). PEA has been shown to have anti-inflammatory, anti-nociceptive, neuro-protective, and anti-convulsant properties.

### SUMMARY OF THE DISCLOSURE

The present disclosure provides pharmaceutical compositions comprising combinations of MEAI, or salts thereof, and N-acylethanolamines, or salts thereof. In some embodiments these combinations comprise specific molar ratios between the respective active agents and/or dosages and may be employed in a variety of methods. Particularly, the present disclosure provides methods for preventing and/or treating a variety of conditions responsive to MEAI treatment, such as alcohol consumption, including alcoholism, eating, tobacco consumption, shopping, or sexual conduct.

The present disclosure provides, in one aspect, a pharmaceutical composition comprising a therapeutically-effective amount of a mixture of MEAI or a salt thereof and at least one N-acylethanolamine or a salt thereof, wherein the molar ratio between the MEAI and the N-acylethanolamine is between about 1:0.2 to about 1:2000.

In certain embodiments, the molar ratio between the MEAI and the N-acylethanolamine is between about 1:0.2 to about 1:5. In certain embodiments, the molar ratio between the MEAI and the N-acylethanolamine is between about 1:0.5 to about 1:2. In certain embodiments, the molar ratio between the MEAI and the N-acylethanolamine is between about 1:15 to about 1:1800. In certain embodiments, the molar ratio between the MEAI and the N-acylethanolamine is between about 1:25 to about 1:450. In certain embodiments, the molar ratio between the MEAI and the N-acylethanolamine is between about 1:50 to about 1:100. In certain embodiments, the molar ratio between the MEAI and the N-acylethanolamine is about 1:50. In certain embodiments, the molar ratio between the MEAI and the N-acylethanolamine is about 1:100. Each possibility represents a separate embodiment of the present disclosure.

In certain embodiments, the pharmaceutical composition comprises about 0.5-10 mg MEAI or a salt thereof. In certain embodiments, the pharmaceutical composition comprises about 1 mg, about 2.5 mg, about 5 mg, or about 10 mg MEAI or a salt thereof. Each possibility represents a separate embodiment of the present disclosure.

In certain embodiments, the pharmaceutical composition comprises about 200-1800 mg N-acylethanolamine or a salt thereof. In certain embodiments, the pharmaceutical composition comprises about 250 mg, about 500 mg, about 750 mg, about 1000 mg or about 1500 mg N-acylethanolamine or a salt thereof. Each possibility represents a separate embodiment of the present disclosure.

In certain embodiments, the N-acylethanolamine is selected from the group consisting of N-palmitoylethanolamine (PEA), Me-palmitoylethanolamide (Me-PEA), palmitoylcyclohexamide, palmitoylbutylamide, palmitoylisopropylamide, oleoylethanolamine (OEA), palmitoylisopropylamide (PIA), salts thereof and any combination thereof. Each possibility represents a separate embodiment of the present disclosure. In certain embodiments, the N-acylethanolamine is PEA or a salt thereof. In certain embodiments, the N-acylethanolamine consists of PEA or a salt thereof. In certain embodiments, the N-acyl ethanol amine consists of PEA.

In certain embodiments, the pharmaceutical composition is formulated for systemic administration. In certain embodiments, the pharmaceutical composition is formulated for oral, oral mucosal, nasal, sublingual, inhalational, topical, rectal, vaginal, parenteral, intravenous, intramuscular, or subcutaneous administration. In certain embodiments, the pharmaceutical composition is formulated for oral, oral mucosal, nasal, or sublingual administration. Each possibility represents a separate embodiment of the present disclosure. In certain embodiments, the pharmaceutical composition is formulated for oral administration. In certain embodiments, the pharmaceutical composition is formulated for oral mucosal administration. In certain embodiments, the pharmaceutical composition is formulated for nasal administration. In certain embodiments, the pharmaceutical composition is formulated for sublingual administration.

The present disclosure further provides, in another aspect, a dosage unit comprising or consisting of the pharmaceutical composition described above.

In certain embodiments, the dosage unit comprises the pharmaceutical composition described above. In certain embodiments, the dosage unit consisting of the pharmaceutical composition described above. In certain embodiments, the dosage unit is formulated as a gel, a powder or a spray. In certain embodiments, the dosage unit is formulated as a gel. In certain embodiments, the dosage unit is formulated as a powder. In certain embodiments, the dosage unit is formulated as a spray.

In certain embodiments, the N-acylethanolamine increases the therapeutic potency of the MEAI compared to the same pharmaceutical composition without the N-acylethanolamine. In certain embodiments, the N-acylethanolamine decreases the required therapeutic dosage of the MEAI compared to the same pharmaceutical composition without the N-acylethanolamine. In certain embodiments, the N-acylethanolamine reduces at least one of the side-effects of the MEAI compared to the same pharmaceutical composition without the N-acylethanolamine. In certain embodiments, the N-acylethanolamine expends the therapeutic window of the MEAI compared to the same pharmaceutical composition without the N-acylethanolamine. In certain embodiments, the PEA or salt thereof increases the therapeutic potency of the THC or salt thereof compared to the same pharmaceutical composition without the PEA or salt thereof. In certain embodiments, the PEA or salt thereof decreases the required therapeutic dosage of the THC or salt thereof compared to the same pharmaceutical composition without the PEA or salt thereof. In certain embodiments, the PEA or salt thereof reduces at least one of the side-effects of the THC or salt thereof compared to the same pharmaceutical composition without the PEA or salt thereof. In certain embodiments, the PEA or salt thereof expends the therapeutic window of the THC or salt thereof compared to the same pharmaceutical composition without the PEA or salt thereof.

In certain embodiments of the methods described above, the MEAI and the N-acylethanolamine are comprised in the same pharmaceutical composition. In certain embodiments of the methods described above, the MEAI and the N-acylethanolamine are comprised in different pharmaceutical compositions.

In certain embodiments of the methods described above, the administration of the MEAI and the N-acylethanolamine is repeated three times a day. In certain embodiments of the methods described above, the administration of the MEAI and the N-acylethanolamine is repeated twice a day. In certain embodiments of the methods described above, the administration of the MEAI and the N-acylethanolamine is repeated once a day. In certain embodiments of the methods described above, the administration of the MEAI and the N-acylethanolamine is repeated once every two days. In certain embodiments of the methods described above, the administration of the MEAI and the N-acylethanolamine is repeated once every three days.

In some embodiments, the present disclosure is directed to a a pharmaceutical composition comprising 5-methoxy-2-aminoindan, or a salt thereof, and an N-acylethanolamine, or a salt thereof, and at least one pharmaceutically acceptable carrier and/or excipient.

In some embodiments, the pharmaceutical composition is a unit dosage form composition. In other embodiments, the pharmaceutical composition is a solid unit dosage form composition. In still other embodiments, the pharmaceutical composition is a liquid unit dosage form composition. In additional embodiments, the pharmaceutical composition is packaged as a single unit dose or as a plurality of single unit doses.

In some embodiments, the unit dosage form comprises from 30 mg to 130 mg of 5-methoxy-2-aminoindan. In some other embodiments, the pharmaceutical composition is formulated for oral administration.

In other embodiments, the disclosure is directed to a method of regulating binge behavior, comprising administrating to a subject in need thereof a therapeutically effective amount of a pharmaceutical composition comprising 5-methoxy-2-aminoindan, or a salt thereof, and an N-acylethanolamine, or a salt thereof, thereby regulating the binge behavior. In some embodiments, the binge behavior is associated with alcohol consumption, eating, tobacco consumption, shopping, or sexual conduct. In other embodiments, the binge behavior is binge drinking.

In further embodiments, the pharmaceutical composition further comprises at least one pharmaceutically acceptable carrier and/or excipient. In other embodiments, the pharmaceutical composition is a free-flowing powder, a tablet, a capsule, a lozenge, a liquid, a liquid concentrate, or a syrup.

In some embodiments, in the methods of the disclosure the pharmaceutical composition is a unit dosage form composition. In other embodiments, the amount of 5-methoxy-2-aminoindan in the unit dosage form ranges from about 30 mg to about 130 mg. In certain embodiments, the amount of 5-methoxy-2-aminoindan is about 70 mg. In some embodiments, the pharmaceutical composition is administered orally.

Further embodiments and the full scope of applicability of the present disclosure will become apparent from the detailed description given hereinafter. However, the detailed description and specific examples, while indicating preferred embodiments of the disclosure, are given by way of illustration only, since various changes and modifications within the spirit and scope of the disclosure will become apparent to those skilled in the art from this detailed description.

### BRIEF DESCRIPTION OF THE FIGURES

The foregoing summary, as well as the following detailed description of the disclosure, will be better understood when read in conjunction with the appended drawings. For the purpose of illustrating the present disclosure, the attached drawings illustrate some, but not all, alternative embodiments. It should be understood, however, that the disclosure is not limited to the precise arrangements and instrumentalities shown. These figures, which are incorporated into and constitute part of the specification, assist in explaining the principles of the disclosures.

FIG. 1 shows food consumption presented as mean fold change of each group out of baseline (i.e. food consumption measured before MEAI treatment)+/−SEM, statistical analysis was performed using Student's T-test of each group vs. control; * p<0.05, *** p<0.001 for mice in Cycle 1 (FIG. 1A) and Cycle 2 (FIG. 1B).

FIGS. 2A and 2B show alcohol consumption of mice in Intermittent Access to 20% Alcohol in two-bottle choice (IA2BC) model. Results of cycle 1 (FIG. 2A) and cycle 2 (FIG. 2B) are presented as mean of alcohol consumption (g/kg/24 h)+SEM, from Day 1 of consumption until Day 47/50. Statistical analysis was performed only on the 6/7 days of treatment, using two-way ANOVA, followed by Bonferroni post-hoc tests for multiple comparisons (*p<0.05; ** p<0.01; *** p<0.001).

FIG. 3 shows alcohol consumption before and after treatment. The results for Cycle 1 are shown in FIG. 3A and for Cycle 2 are shown in FIG. 3B. Results are presented as mean of alcohol consumption (g/kg/24 h)+SEM. Statistical analysis was preformed using two-way ANOVA (repeated measures) followed by Bonferroni posttests to compare replicate means by row. (** p<0.01; *** p<0.001).

FIG. 4 shows the fold change of each group of consumption during 6-7 alcohol days with MEAI/PEA treatment out of its own baseline consumption prior to treatment. Results are presented as mean of fold change+SEM. Mean fold change of all alcohol days together. Statistical analysis was preformed using Student's T-test (*p<0.05).

FIG. 5 shows water consumption of mice in Intermittent Access to 20% Alcohol in two-bottle choice (IA2BC) model. Results of cycle 1 (FIG. 5A) and cycle 2 (FIG. 5B) are presented as mean of water consumption (g/kg/24 h)+SEM, from Day 1 of consumption until Day 47/50.

FIG. 6 shows mean alcohol preference [alcohol consumption g/kg/24 h/(total consumption of fluids g/kg/24 h) for Cycle 1 (FIG. 6A) and Cycle 2 (FIG. 6B). Results are presented as mean±SEM. Statistical analysis was performed using two-way ANOVA, followed by Bonferroni post-hoc tests for multiple comparisons (*p<0.05; ** p<0.01).

FIG. 7 shows alcohol consumption of mice in Intermittent Access to 20% Alcohol in two-bottle choice (IA2BC) model. Results of cycle 1 (FIG. 7A) and cycle 2 (FIG. 7B) are presented as mean of alcohol consumption (g/kg/24 h)+SEM, from Day 22 (6 consumption days before treatment) until Day 47/50 (6/7 consumption days of treatment).

FIGS. 8A and 8B show mean alcohol consumption of cycle 1 and cycle 2 during 6 alcohol days of consumption during MEAI treatment.

FIGS. 9A and 9B show results presented as mean of alcohol consumption (g/kg/24 h)+/−SEM for cycle 1 and cycle groups. Statistical analysis was performed using two-way ANOVA, followed by Bonferroni post-hoc tests for multiple comparisons (*p<0.05; ** p<0.01; *** p<0.001).

FIG. 10 shows fold change in the cycle 1 group of consumption during 6 alcohol days with MEAI treatment to its own consumption prior to MEAI treatment. Results are presented as mean of fold change±SEM. FIG. 10A shows the mean fold change in each of the six alcohol days. FIG. 10B shows mean fold change of all alcohol days together. Statistical analysis of cycle 1 was preformed using paired Student's T-test.

FIG. 11 shows fold change in the cycle 1 group of consumption during 6 alcohol days with MEAI treatment to its own consumption prior to MEAI treatment. Results are presented as mean of fold change±SEM. FIG. 11A shows the mean fold change in each of the six alcohol days. FIG. 11B shows mean fold change of all alcohol days together. Statistical analysis of cycle 2 was preformed using One-way-repeated-measures ANOVA (repeated measures), followed by Bonferroni posttests to compare all means by row (*p<0.05; ** p<0.01; *** p<0.005).

### DETAILED DESCRIPTION OF THE DISCLOSURE

The present disclosure, in some embodiments thereof, relates to binge behavior, and more particularly, but not exclusively, to compositions and methods for regulation of binge behavior, such binge drinking.

The terms “binge”, “binge behavior”, “binge disorder”, “bingeing” and “bingeing behavior” as used herein are interchangeable and relate to non-controlled excessive behavior of indulgence in a variety of activities such as eating, drinking, smoking, drugs use, shopping, sexual conduct, and the like. Binge behavior includes intensive, short episodes of overuse and over consumption of food, alcohol, smoking products, drugs, sweets, sex and the like. Bingeing behaviors are compulsive in style, intensity, habituation, history, motivation, and difficulty to control and remediate. “Compulsive behavior”, as used herein refers to driven behaviors which are often influenced by subconscious desires and motives, as well as strong, uncontrollable, hard-to-tame actions and behaviors which have a predictable pattern.

A subject in need of regulating a binge behavior is typically a subject that experiences episodes of over consumption of the binge's subject (e.g., food, alcohol, smoking, drug use, sweets, sex, shopping, etc.), either on a regular basis, for example, more than once a week, or occasionally, or even rarely, for example once or twice a year, depending on the severity of the binges. Blackout drinkers, for example, that experience a blackout when consuming alcohol even as rarely as twice per year are defined herein as subjects is need of regulating binge drinking. Determining a subject in need of regulating binge behavior can be made, in some cases, according to national or international standards, or by acceptable evaluations by health professionals (e.g., physicians, psychologists, cognitive therapists, social workers, nutritionists, etc., depending on the binge disorder).

In some of any of the embodiments of the present disclosure, binge behavior is associated with alcohol consumption, tobacco consumption, eating, shopping and/or sexual conduct.

The phrase “binge drinking” or “binge drinking disorder” as used herein is determined according national or international definitions as defined by regulation authorities, for example, in line with the definition of binge drinking used by the National Health Service (NHS) and the National Office of Statistics in the United Kingdom (UK), and corresponding services, offices and/or authorities in other countries. The definition of binge drinking used by NHS and the National Office of Statistics describes it as drinking more than double the lower risk guidelines for alcohol in one drinking session, wherein the guidelines advise that people should not regularly drink more than the lower risk guidelines of 3-4 units of alcohol for men (equivalent to a pint and a half of 4% beer (about 852 ml)) and 2-3 units of alcohol for women (equivalent to a 175 ml glass of wine). “Regularly” means drinking every day or most days of the week.

In some embodiments, the method provided herein is for reducing alcohol consumption in a non-binger subject who wishes to control his alcohol consumption, for example, under certain circumstances (for example, during a specific event or time point).

The term “binge drinking regulation” in a broad interpretation refers to controlling the excessive, uncontrolled consumption of alcoholic beverages. Regulating binge drinking relates to reducing the amount of alcohol consumed in a drinking session and/or reducing the number of drinking sessions.

In the context of embodiments of the present disclosure, binge drinking regulation relates to imparting a feeling of satisfaction, satiety or contentedness which discourages binge drinking. Binge drinking regulation as practiced in embodiments of the present disclosure affects a true harm reduction utility by discouraging binge drinking in a way that is easy to implement (by drinking) and non-harmful (pending toxicological validation) to the drinker.

### Definitions

When a range of values is listed, it is intended to encompass each value and sub-range within the range. For example, “C₁-C₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Isomers” means compounds having the same number and kind of atoms, and hence the same molecular weight, but differing with respect to the arrangement or configuration of the atoms in space.

“Stereoisomer” or “optical isomer” mean a stable isomer that has at least one chiral atom or restricted rotation giving rise to perpendicular dissymmetric planes (e.g., certain biphenyls, allenes, and spiro compounds) and can rotate plane-polarized light. Because asymmetric centers and other chemical structure exist in the compounds of the disclosure which may give rise to stereoisomerism, the disclosure contemplates stereoisomers and mixtures thereof. The compounds of the disclosure and their salts include asymmetric carbon atoms and may therefore exist as single stereoisomers, racemates, and as mixtures of enantiomers and diastereomers. Typically, such compounds will be prepared as a racemic mixture. If desired, however, such compounds can be prepared or isolated as pure stereoisomers, i.e., as individual enantiomers or diastereomers, or as stereoisomer-enriched mixtures. As discussed in more detail below, individual stereoisomers of compounds are prepared by synthesis from optically active starting materials containing the desired chiral centers or by preparation of mixtures of enantiomeric products followed by separation or resolution, such as conversion to a mixture of diastereomers followed by separation or recrystallization, chromatographic techniques, use of chiral resolving agents, or direct separation of the enantiomers on chiral chromatographic columns. Starting compounds of particular stereochemistry are either commercially available or are made by the methods described below and resolved by techniques well-known in the art.

It is well-known in the art that the biological and pharmacological activity of a compound is sensitive to the stereochemistry of the compound. Thus, for example, enantiomers often exhibit strikingly different biological activity including differences in pharmacokinetic properties, including metabolism, protein binding, and the like, and pharmacological properties, including the type of activity displayed, the degree of activity, toxicity, and the like. Thus, one skilled in the art will appreciate that one enantiomer may be more active or may exhibit beneficial effects when enriched relative to the other enantiomer or when separated from the other enantiomer. Additionally, one skilled in the art would know how to separate, enrich, or selectively prepare the enantiomers of the compounds of the disclosure from this disclosure and the knowledge of the prior art.

Thus, although the racemic form of drug may be used, it is often less effective than administering an equal amount of enantiomerically pure drug; indeed, in some cases, one enantiomer may be pharmacologically inactive and would merely serve as a simple diluent. For example, although ibuprofen had been previously administered as a racemate, it has been shown that only the S-isomer of ibuprofen is effective as an anti-inflammatory agent (in the case of ibuprofen, however, although the R-isomer is inactive, it is converted in vivo to the S-isomer, thus, the rapidity of action of the racemic form of the drug is less than that of the pure S-isomer). Furthermore, the pharmacological activities of enantiomers may have distinct biological activity. For example, S-penicillamine is a therapeutic agent for chronic arthritis, while R-penicillamine is toxic. Indeed, some purified enantiomers have advantages over the racemates, as it has been reported that purified individual isomers have faster transdermal penetration rates compared to the racemic mixture. See U.S. Pat. Nos. 5,114,946 and 4,818,541.

In some embodiments, the compound is a racemic mixture of(S)- and (R)-isomers. In other embodiments, provided herein is a mixture of compounds wherein individual compounds of the mixture exist predominately in an(S)- or (R)-isomeric configuration. For example, the compound mixture has an(S)-enantiomeric excess of greater than about 55%, about 60%, about 65%, about 70%, about 75%, about 80%, about 85%, about 90%, about 95%, about 96%, about 97%, about 98%, about 99%, about 99.5%, or more. In other embodiments, the compound mixture has an(S)-enantiomeric excess of greater than about 55% to about 99.5%, greater than about 60% to about 99.5%, =greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5%, or more. In other embodiments, the compound mixture has an (R)-enantiomeric purity of greater than about 55%, about 60%, about 65%, about 70%, about 75%, about 80%, about 85%, about 90%, about 95%, about 96%, about 97%, about 98%, about 99%, about 99.5% or more. In some other embodiments, the compound mixture has an (R)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5% or more.

Individual stereoisomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by: (1) attachment of a mixture of enantiomers to a chiral auxiliary, separation of the resulting mixture of diastereomers by recrystallization or chromatography and liberation of the optically pure product from the auxiliary; (2) salt formation employing an optically active resolving agent; or (3) direct separation of the mixture of optical enantiomers on chiral chromatographic columns. Stereoisomeric mixtures can also be resolved into their component stereoisomers by well-known methods, such as chiral-phase gas chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well-known asymmetric synthetic methods.

Thus, if one enantiomer is pharmacologically more active, less toxic, or has a preferred disposition in the body than the other enantiomer, it would be therapeutically more beneficial to administer that enantiomer preferentially. In this way, the patient undergoing treatment would be exposed to a lower total dose of the drug and to a lower dose of an enantiomer that is possibly toxic or an inhibitor of the other enantiomer.

As used herein, nomenclature for compounds including organic compounds, can be given using common names, IUPAC, IUBMB, or CAS recommendations for nomenclature. One of skill in the art can readily ascertain the structure of a compound if given a name, either by systemic reduction of compound structure using naming conventions, or by commercially available software, such as CHEMDRAW™ (Cambridgesoft Corporation, U.S.A.). Chemical names were generated using PerkinElmer ChemDraw® Professional, version 17.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbol “R” or “S,” depending on the configuration of substituents around the stereogenic carbon atom. The present disclosure encompasses various stereoisomers of these compounds and mixtures thereof. Stereoisomers include enantiomers and diastereomers. Mixtures of enantiomers or diastereomers may be designated “(+)” in nomenclature, but the skilled artisan will recognize that a structure may denote a chiral center implicitly. In some embodiments, an enantiomer or stereoisomer may be provided substantially free of the corresponding enantiomer.

The present disclosure provides, in one aspect, a pharmaceutical composition comprising a therapeutically-effective amount of a mixture of MEAI or a salt thereof and at least one N-acylethanolamine or a salt thereof.

The present disclosure provides, in another aspect, a pharmaceutical composition comprising a therapeutically-effective amount of a mixture of MEAI or a salt thereof and at least one N-acylethanolamine or a salt thereof, wherein the molar ratio between the MEAI and the N-acylethanolamine is between about 1:0.2 to about 1:2000.

As used herein, a “pharmaceutical composition” refers to a preparation of the active agents described herein with other chemical components such as physiologically suitable carriers and excipients. The purpose of a pharmaceutical composition is to facilitate administration of a compound to an organism. As used herein, the phrase “pharmaceutically acceptable carrier” refers to a carrier, an excipient or a diluent that does not cause significant irritation to an organism and does not abrogate the biological activity and properties of the administered compound. An adjuvant is included under these phrases.

The term “excipient” as used herein refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, oils such as vegetable oils or fish oils, and polyethylene glycols.

The term “carrier” as used herein refers to a diluent, adjuvant, excipient, or vehicle with which the compound is administered. Such pharmaceutical carriers can be sterile liquids, such as water and oils. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition.

The phrase “pharmaceutically acceptable” as used herein refers to molecular entities and compositions that are physiologically tolerable and do not typically produce an allergic or similar toxicity when administered to an individual. Preferably, and particularly where a formulation is used in humans, the term “pharmaceutically acceptable” may mean approved by a regulatory agency (for example, the U.S. Food and Drug Agency) or listed in a generally recognized pharmacopeia for use in animals (e.g., the U.S. Pharmacopeia).

As used herein, the phrase “pharmaceutically acceptable salt” refers to a charged species of the parent compound and its counter-ion, which is typically used to modify the solubility characteristics of the parent compound and/or to reduce any significant irritation to an organism by the parent compound, while not abrogating the biological activity and properties of the administered compound.

In the context of some embodiments of the present disclosure, a pharmaceutically acceptable salt of the compounds described herein may optionally be an acid addition salt comprising at least one basic (e.g., amine) group of the compound which is in a positively charged form (e.g., an ammonium ion), in combination with at least one counter-ion, derived from the selected acid, that forms a pharmaceutically acceptable salt.

The acid addition salts of the compounds described herein may therefore be complexes formed between one or more basic groups of the drug and one or more equivalents of an acid.

The acid addition salts may include a variety of organic and inorganic acids, such as, but not limited to, hydrochloric acid which affords a hydrochloric acid addition salt, hydrobromic acid which affords a hydrobromic acid addition salt, acetic acid which affords an acetic acid addition salt, ascorbic acid which affords an ascorbic acid addition salt, benzenesulfonic acid which affords a besylate addition salt, camphorsulfonic acid which affords a camphorsulfonic acid addition salt, citric acid which affords a citric acid addition salt, maleic acid which affords a maleic acid addition salt, malic acid which affords a malic acid addition salt, methanesulfonic acid which affords a methanesulfonic acid (mesylate) addition salt, naphthalenesulfonic acid which affords a naphthalenesulfonic acid addition salt, oxalic acid which affords an oxalic acid addition salt, phosphoric acid which affords a phosphoric acid addition salt, toluenesulfonic acid which affords a p-toluenesulfonic acid addition salt, succinic acid which affords a succinic acid addition salt, sulfuric acid which affords a sulfuric acid addition salt, tartaric acid which affords a tartaric acid addition salt and trifluoroacetic acid which affords a trifluoroacetic acid addition salt. Each of these acid addition salts can be either a mono-addition salt or a poly-addition salt, as these terms are defined herein.

In the context of some of the present embodiments, a pharmaceutically acceptable salt of the compounds described herein may optionally be a base addition salt comprising at least one group of the compound which is in a form of an anion, in combination with at least one counter ion (i.e., cation) that forms a pharmaceutically acceptable salt. Examples of suitable cations include metal cations of metals such as, but not limited to, sodium, potassium, magnesium, and calcium or ammonium.

Each of these base addition salts can be either a mono-addition salt or a poly-addition salt, as these terms are defined herein.

Depending on the stoichiometric proportions between the basic or acidic charged group(s) in the compound (e.g., amine group(s)) and the counter-ion in the salt, the acid or base additions salts can be either mono-addition salts or poly-addition salts.

The phrase “mono-addition salt”, as used herein, refers to a salt in which the stoichiometric ratio between the counter-ion and charged form of the compound is 1:1, such that the addition salt includes one molar equivalent of the counter-ion per one molar equivalent of the compound.

The phrase “poly-addition salt”, as used herein, refers to a salt in which the stoichiometric ratio between the counter-ion and the charged form of the compound is greater than 1:1 and is, for example, 2:1, 3:1, 4:1 and so on, such that the addition salt includes two or more molar equivalents of the counter-ion per one molar equivalent of the compound.

Further, in any one of the embodiments described herein, each of the compounds described herein, including the salts thereof, can be in a form of a solvate or a hydrate thereof.

The term “solvate” refers to a complex of variable stoichiometry (e.g., di-, tri-, tetra-, penta-, hexa-, and so on), which is formed by a solute (the 2-aminoindan derivatives described herein) and a solvent, whereby the solvent does not interfere with the biological activity of the solute.

The term “hydrate” refers to a solvate, as defined hereinabove, where the solvent is water.

The term “N-acylethanolamine” as used herein generally refers to a type of fatty acid amide, lipid-derived signaling molecules, formed when one of several types of acyl group is linked to the nitrogen atom of ethanolamine. These amides conceptually can be formed from a fatty acid and ethanolamine with the release of a molecule of water, but the known biological synthesis uses a specific phospholipase D to cleave the phospholipid unit from N-acylphosphatidylethanolamines. The suffixes-amine and -amide in these names each refer to the single nitrogen atom of ethanolamine that links the compound together: it is termed “amine” in ethanolamine because it is considered as a free terminal nitrogen in that subunit, while it is termed “amide” when it is considered in association with the adjacent carbonyl group of the acyl subunit. Names for these compounds may be encountered with either “amide” or “amine” in the present application. The term “ethanolamine” is used in the generic sense and is meant to include mono-ethanolamine, di-ethanolamine, tri-ethanolamine, and mixtures thereof.

The term “derivative” as used herein means a compound whose core structure is the same as, or closely resembles that of an N-acylethanolamine compound, but which has a chemical or physical modification, such as different or additional side groups.

The term “salt” as used herein refers to any form of an active ingredient in which the active ingredient assumes an ionic form and is coupled to a counter ion (a cation or anion) or is in solution. This also includes complexes of the active ingredient with other molecules and ions, in particular complexes which are complexed by ion interaction.

In certain embodiments, the molar ratio between the MEAI and the N-acylethanolamine is between about 1:0.2 to about 1:5. In certain embodiments, the molar ratio between the MEAI and the N-acylethanolamine is between about 1:0.22 to about 1:4.5, about 1:0.25 to about 1:4, between about 1:0.28 to about 1:3.5, between about 1:0.33 to about 1:3, between about 1:0.4 to about 1:2.5, between about 1:0.5 to about 1:2 or about 1:1. Each possibility represents a separate embodiment of the present disclosure.

In certain embodiments, the molar ratio between the MEAI and the N-acylethanolamine is between about 1:15 to about 1:1800. In certain embodiments, the molar ratio between the MEAI and the N-acylethanolamine is between about 1:16 to about 1:1700, about 1:17 to about 1:1600, about 1:18 to about 1:1500, about 1:19 to about 1:1400, about 1:20 to about 1:1300, about 1:21 to about 1:1200, about 1:22 to about 1:1100, about 1:23 to about 1:1000, about 1:24 to about 1:900, about 1:15 to about 1:800, about 1:16 to about 1:700, about 1:17 to about 1:600, about 1:18 to about 1:500, about 1:19 to about 1:490, about 1:20 to about 1:480, about 1:21 to about 1:470, or about 1:22 to about 1:460. Each possibility represents a separate embodiment of the present disclosure. In certain embodiments, the molar ratio between the MEAI and the N-acylethanolamine is between about 1:25 to about 1:450. In certain embodiments, the molar ratio between the MEAI and the N-acylethanolamine is between about 1:10 to about 1:500, about 1:15 to about 1:450, about 1:20 to about 1:400, about 1:25 to about 1:350, about 1:30 to about 1:300, about 1:35 to about 1:250, about 1:40 to about 1:200, or about 1:45 to about 1:150. Each possibility represents a separate embodiment of the present disclosure. In certain embodiments, the molar ratio between the MEAI and the N-acylethanolamine is between about 1:50 to about 1:100. In certain embodiments, the molar ratio between the MEAI and the N-acylethanolamine is about 1:10. In certain embodiments, the molar ratio between the MEAI and the N-acylethanolamine is about 1:20. In certain embodiments, the molar ratio between the MEAI and the N-acylethanolamine is about 1:30. In certain embodiments, the molar ratio between the MEAI and the N-acylethanolamine is about 1:40. In certain embodiments, the molar ratio between the MEAI and the N-acylethanolamine is about 1:50. In certain embodiments, the molar ratio between the MEAI and the N-acylethanolamine is about 1:60. In certain embodiments, the molar ratio between the MEAI and the N-acylethanolamine is about 1:70. In certain embodiments, the molar ratio between the MEAI and the N-acylethanolamine is about 1:80. In certain embodiments, the molar ratio between the MEAI and the N-acylethanolamine is about 1:90. In certain embodiments, the molar ratio between the MEAI and the N-acylethanolamine is about 1:100. In certain embodiments, the molar ratio between the MEAI and the N-acylethanolamine is about 1:110. In certain embodiments, the molar ratio between the MEAI and the N-acylethanolamine is about 1:120. In certain embodiments, the molar ratio between the MEAI and the N-acylethanolamine is about 1:130. In certain embodiments, the molar ratio between the MEAI and the N-acylethanolamine is about 1:140. In certain embodiments, the molar ratio between the MEAI and the N-acylethanolamine is about 1:150. In certain embodiments, the molar ratio between the MEAI and the N-acylethanolamine is about 1:160. In certain embodiments, the molar ratio between the MEAI and the N-acylethanolamine is about 1:170. In certain embodiments, the molar ratio between the MEAI and the N-acylethanolamine is about 1:180. In certain embodiments, the molar ratio between the MEAI and the N-acylethanolamine is about 1:190. In certain embodiments, the molar ratio between the MEAI and the N-acylethanolamine is about 1:200. In certain embodiments, the molar ratio between the MEAI and the N-acylethanolamine is at least about 1:10, at least about 1:20, at least about 1:30, at least about 1:40, at least about 1:50, at least about 1:60, at least about 1:70, at least about 1:80, at least about 1:90, or at least about 1:100. Each possibility represents a separate embodiment of the present disclosure.

In certain embodiments, the pharmaceutical composition comprises about 0.5-10 mg MEAI or a salt thereof. In certain embodiments, the pharmaceutical composition comprises about 1-9.5 mg, about 1.5-9 mg, about 2-8.5 mg, about 2.5-8 mg, about 3-7.5 mg, about 3.5-7 mg, about 4-6.5 mg, about 4.5-6 mg or about 5-5.5 mg MEAI or a salt thereof. In certain embodiments, the pharmaceutical composition comprises about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg or about 10 mg MEAI or a salt thereof. Each possibility represents a separate embodiment of the present disclosure. In certain embodiments, the pharmaceutical composition comprises less than about 0.5 mg, less than about 1 mg, less than about 1.5 mg, less than about 2 mg, less than about 2.5 mg, less than about 3 mg, less than about 3.5 mg, less than about 4 mg, less than about 4.5 mg, less than about 5 mg, less than about 5.5 mg, less than about 6 mg, less than about 6.5 mg, less than about 7 mg, less than about 7.5 mg, less than about 8 mg, less than about 8.5 mg, less than about 9 mg, less than about 9.5 mg or about 10 mg MEAI or a salt thereof. Each possibility represents a separate embodiment of the present disclosure. In certain embodiments, the pharmaceutical composition comprises about 0.5 mg to about 1 mg, about 0.5 mg to about 1.5 mg, about 0.5 mg to about 2 mg, about 0.5 mg to about 2.5 mg, about 0.5 mg to about 3 mg, about 0.5 mg to about 3.5 mg, about 0.5 mg to about 4 mg, about 0.5 mg to about 4.5 mg, about 0.5 mg to about 5 mg, about 0.5 mg to about 5.5 mg, about 0.5 mg to about 6 mg, about 0.5 mg to about 6.5 mg, about 0.5 mg to about 7 mg, about 0.5 mg to about 7.5 mg, about 0.5 mg to about 8 mg, about 0.5 mg to about 8.5 mg, about 0.5 mg to about 9 mg or about 0.5 mg to about 9.5 mg MEAI or a salt thereof. Each possibility represents a separate embodiment of the present disclosure.

In certain embodiments, the pharmaceutical composition comprises about 200-1800 mg N-acylethanolamine or a salt thereof. In certain embodiments, the pharmaceutical composition comprises about 250-1550 mg, about 300-1200 mg, about 350-950 mg, about 400-700 mg, about 450-600 mg or about 500-550 mg N-acylethanolamine or a salt thereof. Each possibility represents a separate embodiment of the present disclosure. In certain embodiments, the pharmaceutical composition comprises at least about 50 mg, at least about 100 mg, at least about 150 mg, at least about 200 mg, at least about 250 mg, at least about 300 mg, at least about 350 mg, at least about 400, at least about 450 mg, at least about 500 mg, at least about 550 mg, at least about 600 mg, at least about 650 mg, at least about 700 mg, at least about 750 mg, at least about 800 mg, at least about 850 mg, at least about 900 mg, at least about 950 mg, at least about 1000 mg, at least about 1050 mg, at least about 1100 mg, at least about 1150 mg, at least about 1200 mg, at least about 1250 mg, at least about 1300 mg, at least about 1350 mg, at least about 1400 mg, at least about 1450 mg, at least about 1500 mg, at least about 1550 mg, at least about 1600 mg, at least about 1650 mg, at least about 1700 mg, at least about 1750 mg or at least about 1800 mg N-acylethanolamine or a salt thereof. In certain embodiments, the pharmaceutical composition comprises about 50 mg, about 100 mg, about 150 mg, about 200 mg, about 250 mg, about 300 mg, about 350 mg, about 400, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg or about 1800 mg N-acylethanolamine or a salt thereof. Each possibility represents a separate embodiment of the present disclosure.

In certain embodiments, the N-acylcthanolamine is selected from the group consisting of N-palmitoylethanolamine (PEA), Me-palmitoylethanolamide (Me-PEA), palmitoylcyclohexamide, palmitoylbutylamide, palmitoylisopropylamide, oleoylethanolamine (OEA), palmitoylisopropylamide (PIA), salts thereof and any combination thereof. Each possibility represents a separate embodiment of the present disclosure. In certain embodiments, the N-acylethanolamine is PEA or a salt thereof. In certain embodiments, the N-acylethanolamine consists of PEA or a salt thereof. In certain embodiments, the N-acylethanolamine consists of PEA.

In certain embodiments, the pharmaceutical composition is formulated for systemic administration. In certain embodiments, the pharmaceutical composition is formulated for oral, oral mucosal, nasal, sublingual, inhalational, topical, rectal, vaginal, parenteral, intravenous, intramuscular, or subcutaneous administration. In certain embodiments, the pharmaceutical composition is formulated for oral, oral mucosal, nasal, or sublingual administration. Each possibility represents a separate embodiment of the present disclosure. In certain embodiments, the pharmaceutical composition is formulated for oral administration. In certain embodiments, the pharmaceutical composition is formulated for oral mucosal administration. In certain embodiments, the pharmaceutical composition is formulated for nasal administration. In certain embodiments, the pharmaceutical composition is formulated for sublingual administration.

Techniques for formulation and administration of drugs are well known in the art, and may be found, e.g. in “Remington's Pharmaceutical Sciences,” Mack Publishing Co., Easton, Pa. Pharmaceutical compositions of the present disclosure may be manufactured by processes well known in the art, e.g., by means of conventional mixing, dissolving, granulating, dragee-making, levigating, emulsifying, encapsulating, entrapping, or lyophilizing processes.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries as desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, and sodium carbomethylcellulose; and/or physiologically acceptable polymers such as polyvinylpyrrolidone (PVP). If desired, disintegrating agents, such as cross-linked polyvinyl pyrrolidone, agar, or alginic acid or a salt thereof, such as sodium alginate, may be added.

The term “oral administration” refers to any method of administration in which an active agent can be administered by swallowing, chewing, sucking, or drinking an oral dosage form. Examples of solid dosage forms include conventional tablets, multi-layer tablets, capsules, caplets, etc., which do not substantially release the drug in the mouth or in the oral cavity.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions that can be used orally include stiff or soft, sealed capsules made of gelatin and a plasticizer, such as glycerol or sorbitol. The capsules may contain the active ingredients in admixture with filler such as lactose, binders such as starches, lubricants such as talc or magnesium stearate, and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration. For buccal and sublingual administration, the compositions may take the form of tablets or lozenges formulated in conventional manner or in adhesive carriers. Alternatively, the active ingredient may be in powder form for constitution with a suitable vehicle, e.g., a sterile, pyrogen-free, water-based solution, before use.

Pharmaceutical compositions suitable for use in the context of the present disclosure include compositions wherein the active ingredients are contained in an amount effective to achieve the intended purpose. More specifically, a “therapeutically effective amount” means an amount of active ingredients effective to prevent, alleviate, or ameliorate symptoms or side effects of a disease or disorder, or prolong the survival of the subject being treated. Determination of a therapeutically effective amount is well within the capability of those skilled in the art, especially in light of the detailed disclosure provided herein. More specifically, a “therapeutically effective amount of a mixture” means an amount of at least two active ingredients, wherein each one of the active ingredients independently may not be in a therapeutically effective amount or wherein both of the active ingredients may not be in a therapeutically effective amount, the mixture is nevertheless effective to prevent, alleviate, or ameliorate symptoms or side effects of a disease or disorder, or prolong the survival of the subject being treated. The term “mixture” as used herein refers to a non-covalent combination of two molecules.

For any preparation used in the methods of the disclosure, the dosage or the therapeutically effective amount can be estimated initially from in vitro, in vivo and cell culture assays. For example, a dose can be formulated in animal models to achieve a desired concentration or titer. Such information can be used to more accurately determine useful doses in humans. The dosage of each compound of the claimed combinations depends on several factors, including: the administration method, the disease to be treated, the severity of the disease, whether the disease is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect dosage used. Continuous daily dosing may not be required; a therapeutic regimen may require cycles, during which time a drug is not administered, or therapy may be provided on an as-needed basis during periods of acute disease worsening. Dosage escalation may or may not be required; a therapeutic regimen may require reduction in medication dosage. Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration, and dosage can be chosen by the individual physician in view of the patient's condition (See, e.g., Fingl, E. et al. (1975), “The Pharmacological Basis of Therapeutics,” Ch. 1, p. 1). Depending on the severity and responsiveness of the condition to be treated, dosing can be of a single or a plurality of administrations, with course of treatment lasting from several days to several weeks, or until cure is affected or diminution of the disease state is achieved.

The present disclosure further provides, in another aspect, a dosage unit comprising or consisting of the pharmaceutical composition described above.

In certain embodiments, the dosage unit comprises the pharmaceutical composition described above. In certain embodiments, the dosage unit consisting of the pharmaceutical composition described above. In certain embodiments, the dosage unit is formulated as a gel, a powder or a spray. In certain embodiments, the dosage unit is formulated as a gel. In certain embodiments, the dosage unit is formulated as a powder. In certain embodiments, the dosage unit is formulated as a spray.

The present disclosure further provides, in another aspect, a pharmaceutical composition or a dosage unit as described above for use in a method for preventing or treating a condition amenable to prevention or treatment by at least one MEAI.

The term “treating” as used herein, includes, but is not limited to, any one or more of the following: abrogating, ameliorating, inhibiting, attenuating, blocking, suppressing, reducing, delaying, halting, alleviating or preventing one or more symptoms or side effects of the diseases or conditions of the disclosure.

The term “acute” refers to a condition with a relatively short, severe course.

The term “chronic” as used herein means that the length of time of the diseases or conditions of the disclosure can be weeks, months, or possibly years. The intensity of the diseases or conditions can differentiate according to various conditions such as patient age, temperature, season, type of disease, etc.

The term “about” as used herein in relation to a value, a plurality of values or a range of values defined by a lowest and highest values means a value which is 10% lower and/or higher than the corresponding value, plurality of values or range of values. For example, the phrase “about 1” means “0.9 to 1.1”, the phrase “about 1 or 2” means “0.9 to 1.1 or 1.8 to 2.2”, and the phrase “about 1 to about 2” means “0.9 to 2.2”.

The terms “comprises”, “comprising”, “includes”, “including”, “having” and their conjugates mean “including but not limited to.”

The term “consisting of” means “including and limited to”.

The term “consisting essentially of” means that the composition, method or microcapsules may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferable.

Data obtained from the cell culture assays or animal studies can be used in formulating a range of dosage for use in humans. Therapeutically effective dosages achieved in one animal model may be converted for use in another animal, including humans, using conversion factors known in the art (see, e.g., Freireich et al., Cancer Chemother. Reports 50 (4): 219-244 (1966) and the following Table for Equivalent Surface Area Dosage Factors).

TABLE 1
Equivalent Surface Area Dosage Factors.

To:

| | Mouse | Rat | Monkey | Dog | Human |
|---|---|---|---|---|---|
| From: | (20 g) | (150 g) | (3.5 kg) | (8 kg) | (60 kg) |
| Mouse | 1 | ½ | ¼ | ⅙ | 1/12 |
| Rat | 2 | 1 | ½ | ¼ | 1/7 |
| Monkey | 4 | 2 | 1 | ⅗ | ⅓ |
| Dog | 6 | 4 | ⅗ | 1 | ½ |
| Human | 12 | 7 | 3 | 2 | 1 |

The dosage of such compounds lies preferably within a range of circulating concentrations that include the ED₅₀ with little or no toxicity. The dosage may vary within this range depending upon the dosage form employed and the route of administration utilized. Generally, a therapeutically effective amount may vary with the subject's age, condition, and gender, as well as the severity of the medical condition in the subject. The dosage may be determined by a physician and adjusted, as necessary, to suit observed effects of the treatment.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trials including first-in-human, dose ranging and efficacy trials, in healthy patients and/or those suffering from a given disorder, may be completed according to methods well known in the clinical and medical arts.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure is not limited to the particular embodiment disclosed, but that the present disclosure will include all embodiments falling within the scope of the appended claims.

The following examples are presented in order to more fully illustrate some embodiments of the disclosure. They should, in no way be construed, however, as limiting the broad scope of the disclosure.

### EXAMPLES

### Example 1: Evaluation of the Efficacy of MEAI and PEA in Binge Alcohol Consumption in Mice

Alcohol consumption and alcoholism present a growing problem worldwide, which also believed to overtaken tobacco in terms of overall health and social care costs. Excessive and/or prolonged alcohol consumption may have some undesired physiological and psychological, including short-term effects such as gastric irritation, anxiety disorders and other excitable states, and longer-term effects such as cirrhosis, fatty liver disease, cardiomyopathy and dementia.

MEAI (5-methoxy-2-aminoindane or 5-MeO-Al) is a derivative of 2-Aminoindan, useful as binge regulators or binge mitigation agents that impart a feeling of satisfaction, satiety or contentedness and regulate (e.g., discourage) binge behavior such as binge drinking. Administration of an effective amount of the binge regulating agent, whether alone or in combination with an alcoholic beverage, to a subject is expected to reduce the amount of alcohol consumed in a binge drinking, and to prevent many of the adverse effects associated with excessive alcohol consumption. This relates particularly to short-term effects such as heavy intoxication that leads to serious consequences such as accidents and uncontrolled violent behavior with subsequent medical complications, as well as gastric irritation, anxiety disorders and other excitable states.

The objective of this study was to evaluate the efficacy of MEAI in reducing binge alcohol consumption in mice, at several MEAI doses and in combination with Palmitoylethanolamide (PEA).

The principle of the study is based on Intermittent access to 20% Alcohol in two-bottle choice (IA2BC) model in mice. The mice were provided during the whole Study with two bottles: one bottle of water, and another bottle of 20% ethanol solution, which were removed and replaced after 24 hours with another buttle of water, for additional 24 hours. In total, the mice were subjected to 20% ethanol 24 hours, three times a week, for seven-eight weeks (21-22 alcohol consumption days).

The alcohol consumption was measured after each 24 of alcohol exposure (by weighing the alcohol buttle before and after). Following four weeks of intermittent access to 20% alcohol the mice were allocated to groups according to their alcohol consumption, to achieve balanced groups. To examine the effect of treatment in reducing alcohol consumption, mice were treated with the Test Item/s or Vehicle daily during weeks 6-7/8.

Vehicle and test formulations were prepared as set forth in Table 2.

TABLE 2
Formulation of the Test Items and Vehicle*

| | | | | | | | | | | x No. of | | |
|---|---|---|---|---|---|---|---|---|---|---|---|---|
| | | Dose | Dose | Dose | per Animal | | No of | x No of | | Administrations | | Total |
| | | Level | Volume | Solution | Weight | | Animals | Animals | | (14) | | Amount |
| Test Item | Gr # | mg/kg | mL/kg | mg/mL | mL | mg | X2 | mL | mg | mL | mg | (mg) |
| MEAL | 2F, 8F | 20 | 10 | 2 | 0.25 | 0.5 | 18 | 4.5 | 9 | 54 | 126 | 1386 |
| | 3F | 40 | | 4 | 0.25 | 1.0 | 18 | 4.5 | 18 | 54 | 252 | |
| | 4F, 9F | 10 | | 6 | 0.25 | 1.5 | 18 | 4.5 | 27.0 | 54 | 378 | |
| | 5F, 10F | 100 | | 10 | 0.25 | 2.5 | 18 | 4.5 | 45 | 54 | 630 | |
| PEA | 7F | 25 | 5 | 5 | 0.125 | 0.625 | 10 | 1.25 | 6.25 | 17.5 | 87.5 | 245 |
| | 8F | 25 | | 5 | 0.125 | 0.625 | 18 | 2.25 | 11.25 | 31.5 | 157.5 | |
| | 9F | 25 | | 5 | 0.125 | 0.625 | 18 | 2.25 | 11.25 | 31.5 | 157.5 | |
| | 10F | 25 | | 5 | 0.125 | 0.625 | 18 | 2.25 | 11.25 | 31.5 | 157.5 | |

“F”—Female;
“Gr”—Group;
“No.”—Number

### Formulation Preparation:

Vehicle 1: was prepared from water for injection.

Vehicle 2: solution was prepared from Ethanol: Kolliphor: Water for injection (1:1:18, respectively).

Test Item 1 (MEAI): was supplied as a powder and was dissolved using Vehicle 1 at several dilutions according to Table 2.

Test Item 2 (PEA): was supplied as a powder and was dissolved using Vehicle 2 at 5 mg/mL according to Table 2.

### Experimental Model

Animals: C57BL/6 mice, female, 7-8 weeks at study initiation (Envigo RMS Ltd., Israel). The minimum and maximum initial body weight within each group did not exceed+20% of group's mean weight.

Animal handling was performed according to guidelines of the National Institute of Health (NIH) and the Association for Assessment and Accreditation of Laboratory Animal Care (AAALAC). For this study, mice were housed in filtered cages (up to 5 per cage) measuring 36.5×20.7×13 cm with stainless steel top grill facilitating pelleted food and drinking water in plastic bottle; bedding: steam sterilized clean paddy husk (Envigo, Teklad, Laboratory grade, Sani-chips). Bedding material was changed along with the cage at least once a week.

The mice were fed ad libitum a commercial rodent diet (Teklad Certified Global 18% Protein Diet, Envigo cat #2018SC). Mice had free access to sterilized and acidified drinking water (pH between 2.5 and 3.5) obtained from the municipality supply

The mice were housed under standard laboratory conditions, air conditioned and filtered (HEPA F6/6) with adequate fresh air supply (Minimum 15 air changes/hour). The mice were kept in a climate-controlled environment. Temperatures range was 18-24° C. and relative humidity range 30-70% with a reversed 12 hours light and 12 hours dark cycle (6 PM/6 AM).

Animals were randomly allocated to cages on the day of reception, or if required according to weight.

The mice were examined by the Attending Veterinarian (AV) to determine they were fit for the study before the experimental phase initiation. Animals were inspected daily for any signs of morbidity of mortality.

### Experimental Design and Conditions

The administration of pain reducing agents might interfere with the study, therefore no pain reducing drugs were used.

Animals with weight reduction of 10% of the initial weight received wet food at the bottom of the cage and, as needed, SC injection of saline, and were weighed every day.

Animals found in a moribund condition and animals showing severe prolonged pain and enduring signs of severe prolonged distress were humanely euthanized according to the details in Ethics Committee approval. Animals showing a decrease of body weight greater than 20% from initial body weight determination or 10% from the last consecutive daily measurement were humanely euthanized as well.

First dosing day was assigned “Day 1” and termination day was “Day 50” for Cycle 1 and Day 52 for Cycle 2.

The study was conducted in two cycles. The first cycle included five groups: four were treated with elevated concentrations of MEAI and control groups of ‘Water for Injection’-Vehicle 1. In the second cycle, three additional groups of mice with a combination of MEAI+PEA (in elevated dilutions of MEAI), another group of PEA+Vehicle 1 treated mice and another group of Vehicle 2, which includes the solvent of PEA (Ethanol: Kolliphor: Water at 1:1:18) were treated.

In both cycles, during the first five weeks the mice received only alcohol (three times a week), to establish the IA2BC model and the binge drinking. During the last two weeks, in addition to alcohol treatment, given every other day, the mice were administered everyday with the Test Items/Vehicles (MEAI, PEA) to examine its effect on the alcohol consumption. The dosing was performed right before the alcohol addition (15-30 min). Five additional mice, 12 weeks of age, were not treated with alcohol and served as naïve mice for histology controls.

Each Test Item and vehicle formulation was administered to 9 or 5 mice per group, according to Table 3. Study timeline was performed according to Table 4.

TABLE 3
Group Allocation

| | | | Dose | Dose | Route |
|---|---|---|---|---|---|
| | No. of | | Level | Volume | of |
| Group | Animals | Treatment | (mg/kg) | (mL/kg) | administration |
| 1F | 9 | Vehicle 1 | 0 | 10 | PO |
| 2F | 9 | MEAI | 20 | | |
| 3F | 9 | MEAI | 40 | | |
| 4F | 9 | MEAI | 60 | | |
| 5F | 9 | MEAI | 100 | | |
| 6F | 5 | Vehicle 1 + Vehicle 2 | 0/0 | 10 (MEAI) | PO (MEAI) and |
| 7F | 5 | Vehicle 1 + PEA | 0/25 | 5 (PEA) | IP (PEA) |
| 8F | 9 | MEAI + PEA | 20/25 | | |
| 9F | 9 | MEAI + PEA | 60/25 | | |
| 10F | 9 | MEAI + PEA | 100/25 | | |

TABLE 4
Study Timeline*

| Study | Food | | Body | Clinical | Ethanol | |
|---|---|---|---|---|---|---|
| Day | Consumption | Procedure | Weight^{#} | Observations | Addition | Termination |
| Accl′ | | | ✓ | | | Terminal bleeding |
| 1 to 35 | | | Three | Three | On Day | (~0.5 mL from |
| 24 | FC-in | | times a | times a | 1 and | submandibular vein |
| 25 | FC-out, FC-in | | week | week | three | from five mice of each |
| 26 | FC-Out | | | | times a | group) for |
| 36-49/51 | Three times a | PO/PO + IP | | | week | bioanalytical |
| | week during | Dosing | | | | evaluation of the |
| | ethanol | | | | | plasma |
| | addition, and | | | | | Necropsy: Brain, |
| | FC--in on last | | | | | Kidneys and Liver |
| | dosing day | | | | | from same mice was |
| 50/52 | FC-out | Termination | ✓ | ✓ | | snaped frozen and |
| | | | | | | kept at −80. |
| | | | | | | Liver, Kidneys, |
| | | | | | | Heart, Pancreas, |
| | | | | | | Brain, Spleen, Lungs |
| | | | | | | and thyroid were |
| | | | | | | harvested and fixed in |
| | | | | | | Formalin from the rest |
| | | | | | | of the mice. |

^{#}In Cycle 1 Termination on Day 50. In Cycle 2 Termination on Day 52.

### Tests and Evaluations:

Morbidity and mortality checks were performed daily. Animals that were sacrificed during the study were considered for the interpretation of study results as animals that died during the study. In case of mortality before the study's scheduled termination, gross pathology evaluation was performed as close as possible to the time of death. The time of death was recorded as precisely as possible.

Body weight was recorded upon arrival, before study initiation, three times a week thereafter and at termination.

The animals were observed for clinical symptoms starting on Day 1, and three times a week thereafter until study termination. Observations documented any irregularities, such as changes in local injection site, skin, fur, eyes, mucous membranes, respiratory, occurrence of secretions and excretions (e.g. diarrhea) and autonomic activity (e.g. lacrimation, salivation, piloerection, pupil size, unusual respiratory pattern). Changes in gait, posture, and response to handling, as the presence of bizarre behavior, tremors, convulsions, sleep, and coma were also included. All observed abnormalities, toxic signs, moribund condition, and pre-terminal deaths were recorded.

Animal food consumption was performed twice before first dosing (during alcohol addition, and without alcohol addition), during dosing period at each alcohol addition (six times in total) and before termination (without alcohol addition).

The Food consumption was recorded in each cage for a 18±2 hours period. Determinations of food consumption (calculated value) were based on a provided diet placed in hoppers and the remaining unused diet weighed.

Animal alcohol and water consumption was recorded for each cage and was done for a 24±2 hours period starting at Day 1, three times a week thereafter until the end of the Study.

At termination, five animals from groups 1-10 (two animals from groups 6F and 7F) were (approximately 0.5 mL) from the submandibular vein and sacrificed as specified according to the Ethics Committee approval. The blood was collected into K3-EDTA tubes. Plasma was separated and stored at (−60° C.) to (−90° C.) for future bioanalytical evaluation. Following terminal bleeding, the brain, kidneys, and liver from the same five mice of each group, were incised and snap-frozen for future bioanalytical evaluation (in groups 6F and 7F only two mice will be taken for future bioanalytical evaluation).

Organs from the rest of the animals from each group were harvested and fixed in buffered 4% formaldehyde for possible future histology evaluation (liver, kidneys, heart, pancreas, brain, spleen, lungs, and thyroid).

Numerical results were given as means and standard deviations or standard errors. Whenever applicable, descriptive statistics and group comparisons of data were performed using appropriate statistical analysis programs (such as GraphPad Prism version 5.02 for Windows, GraphPad Software, San Diego California USA). A probability of 5% (p<0.05) was regarded as statistically significant.

Body weight (BW) was monitored every other day in all animals of all groups. Two-way ANOVA revealed no significant changes in BW of all groups compared to control group (1F in cycle 1, and 6F and 7F in cycle 2), except from group 4F (MEAI mid dose), which was significantly lower than the control group only during Day 40 of cycle 1 (*p<0.05).

The animals were evaluated for macroscopic findings during gross pathology. The pathology findings that were detected in the liver, kidneys, heart and spleen (bright color and/or enlarged) were observed in all groups including the vehicle treated (groups 1F, 6F and 7F). All groups were treated with alcohol, suggesting that these were most likely related to the alcohol consumption and not to the MEAI treatment.

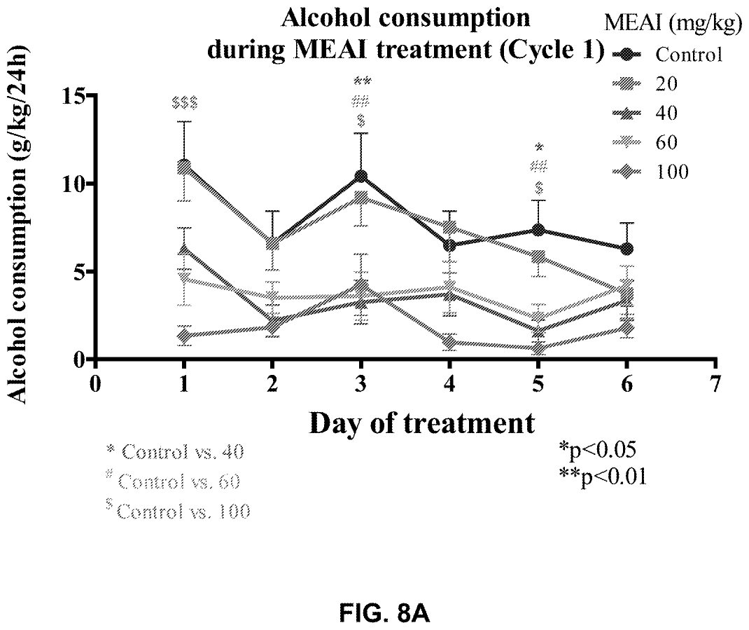

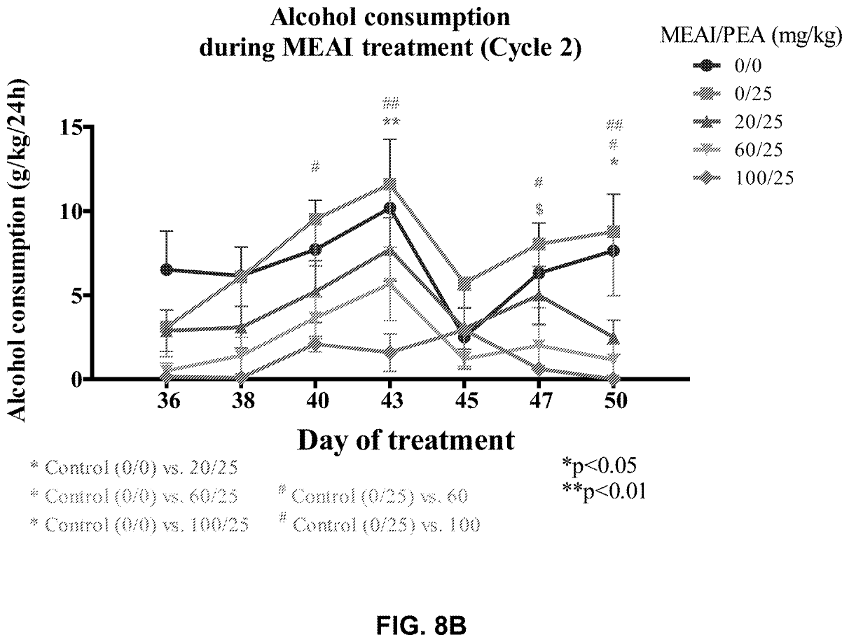

In addition, white dots were found in mice from groups 9F and 10F in cycle 2, but not cycle 1, in organs such as the pancreas (see appendix section FIG. 8), liver and peritoneum, sub-dermal and fat tissues. It is possible that these white dots are accumulation of the PEA.

### Results:

Results showed a significant dose dependent effect for the MEAI treatment in reducing alcohol consumption of the treated animals, with additive effect for the PEA treatment at the lower MEAI doses. There was no significant difference between the animals' body weight, and the food consumption was also not affected by the MEAI treatment.

During the study, mortality was observed in four animals from the high doses treated groups however, it is important to take into consideration that these animals were treated with MEAI following a prolonged period of alcohol consumption. Supporting evidence to the harmful effect of alcohol consumption can be found in the gross pathology analysis which exhibited macroscopic findings mostly in the liver, kidneys, heart, and spleen, of all groups including the control.

Based on the above findings it can be concluded that MEAI exhibited a suppressive effect on mice alcohol consumption in the IA2BC model, and that PEA improved this effect at the lower MEAI doses.

The animals were evaluated for macroscopic findings during gross pathology. The pathology findings that were detected in the liver, kidneys, heart and spleen (bright color and/or enlarged), were observed in all groups including the vehicle treated (groups 1F, 6F and 7F). All groups were treated with alcohol, suggesting that these were most likely related to the alcohol consumption and not to the MEAI treatment.

In addition, white dots were found in mice from groups 9F and 10F in cycle 2, but not cycle 1, in organs such as the pancreas, liver and peritoneum, sub-dermal and fat tissues. It is possible that these white dots are accumulation of the PEA.

### Food Consumption:

Food consumption was monitored per each mouse in each individual cage for 24 h during Alcohol addition time, by measuring the weight of food before and after 24 hours. The consumption of food was measured during all MEAI treatment days, in both cycles. The mean value for each group during each consumption day was calculated as fold change from baseline (i.e. food consumption measured before MEAI treatment), per each animal. The fold change values of each MEAI/PEA treated group was compared to the vehicle groups (1F/6F/7F) and statistical analysis was performed.

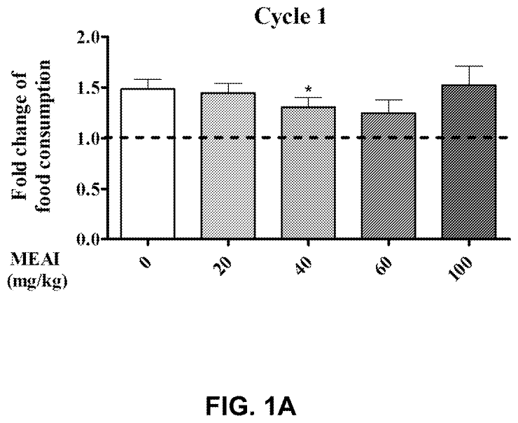

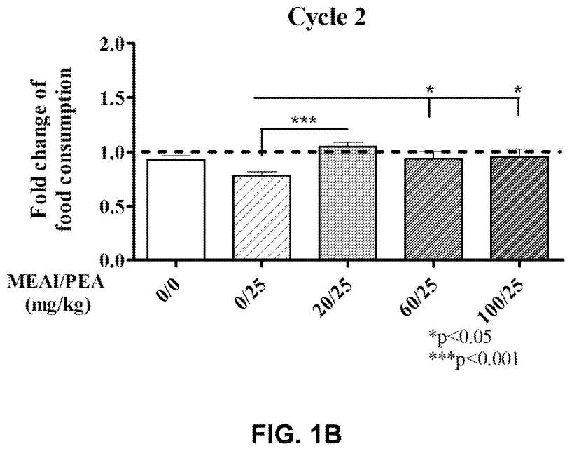

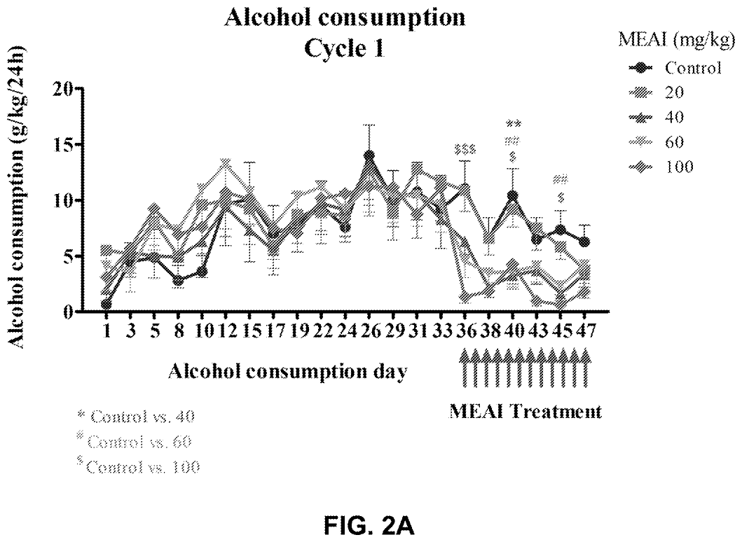

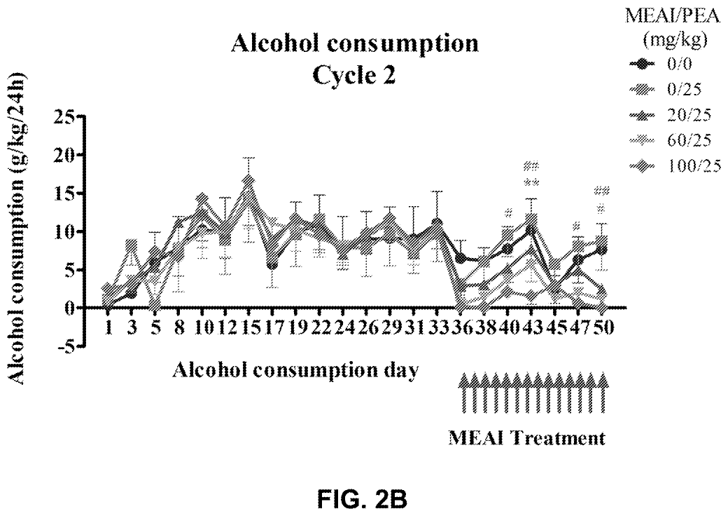

As shown in FIG. 1A, in cycle 1, all groups presented elevation in food consumption as compared to their own baseline (>1.0 fold change). The fold change of group 3F (MEAI 40 mg/kg) was significantly lower compared to the control group 1F (MEAI 0) (*p<0.05). The fold change of group 4F (MEAI 60 mg/kg) was lower than the control, however, not significant. Groups 2F (MEAI 20 mg/kg) and 5F (MEAI 100 mg/kg) were similar to the control Group 1F.

In cycle 2, there was no change in the food consumption in any of the groups compared to their own baseline, except from Control Group 7F (MEAI/PEA 0/25 mg/kg), which was reduced by 22 percent to 0.78±0.03 fold. See FIG. 1B. The other groups, which showed no changed compared to their own baseline level showed significantly higher food consumption as compared to Group 7F (Group 8F MEAI/PEA 20/25 mg/kg, *** p<0.001; Groups 9F and 10F MEAI/PEA 60/25 mg/kg and 100/25 mg/kg, *p<0.05).

### Alcohol Consumption:

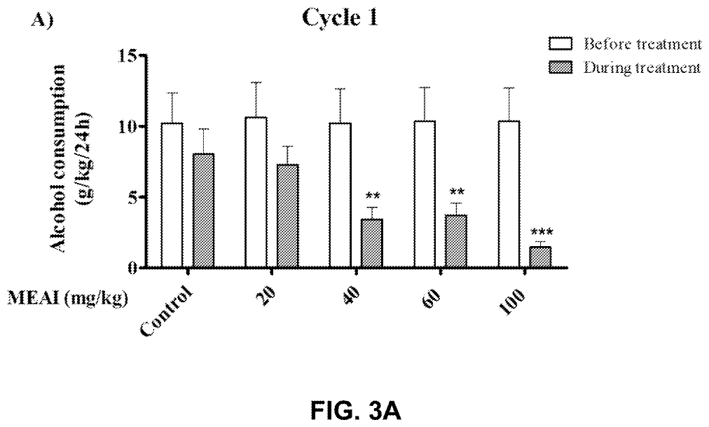

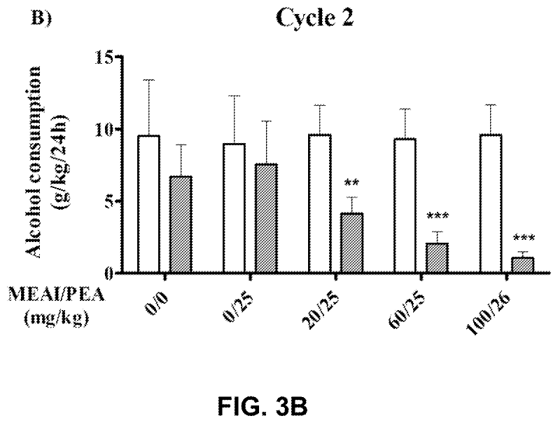

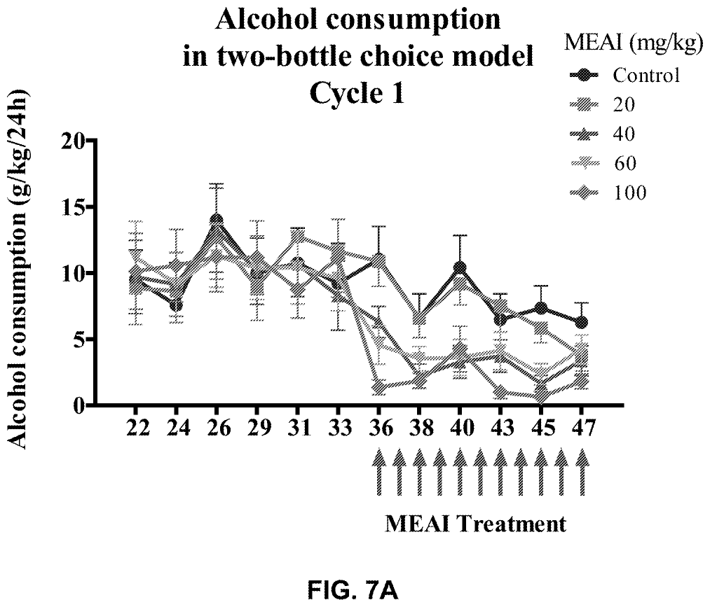

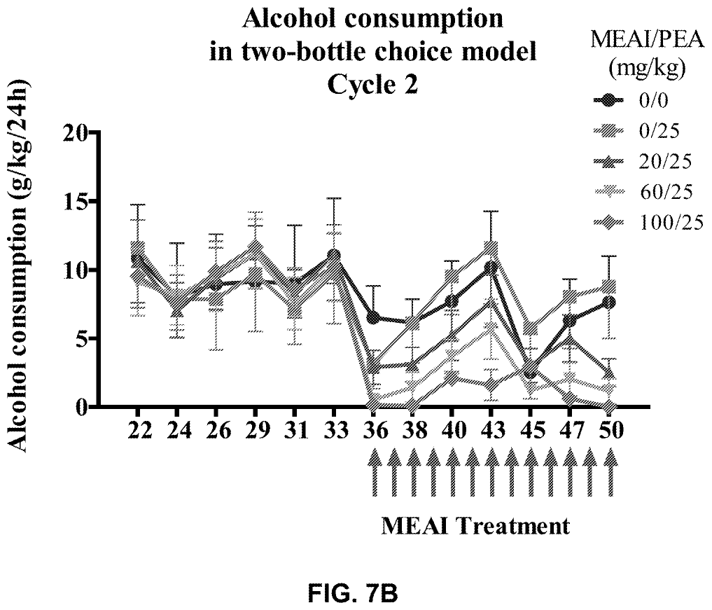

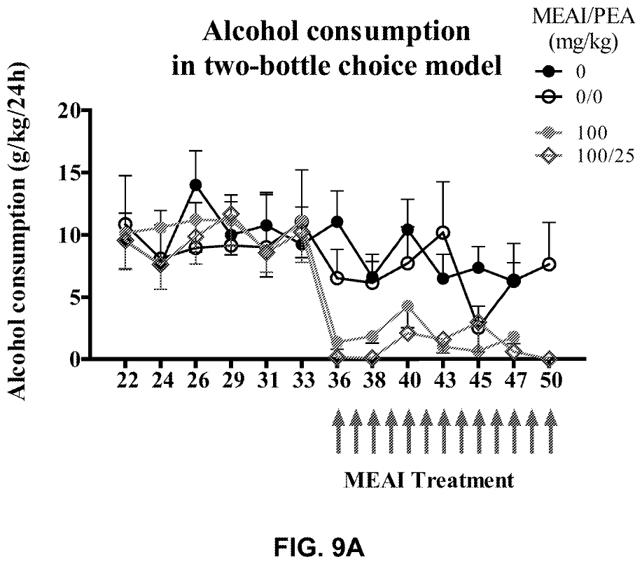

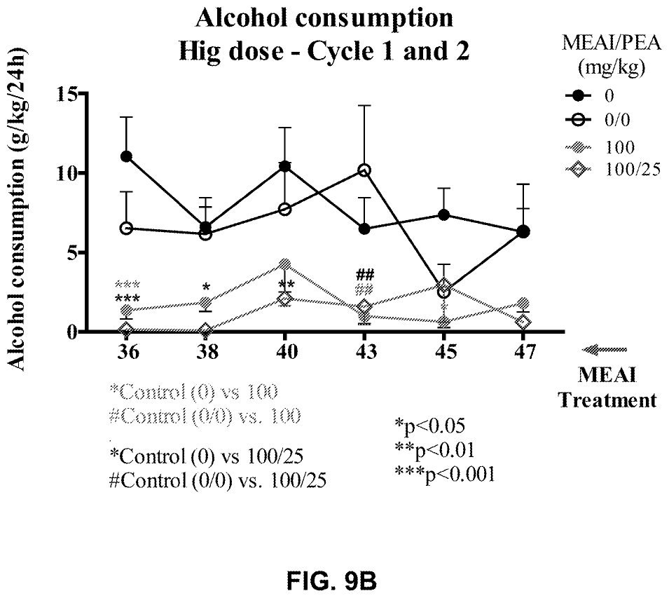

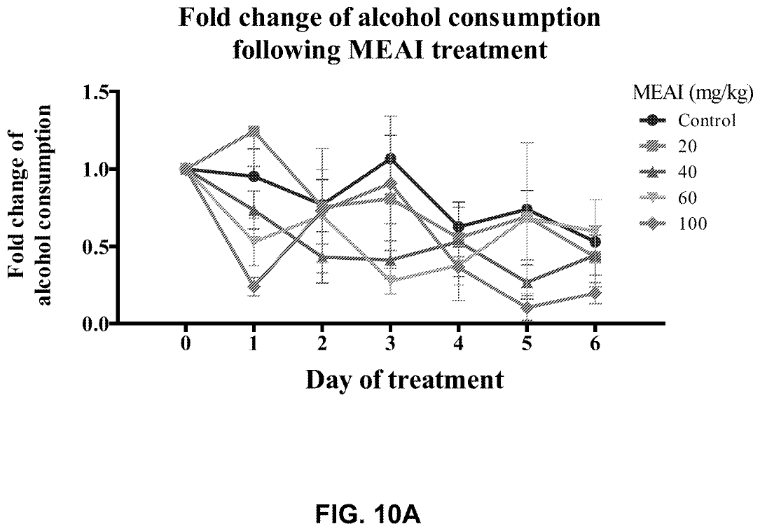

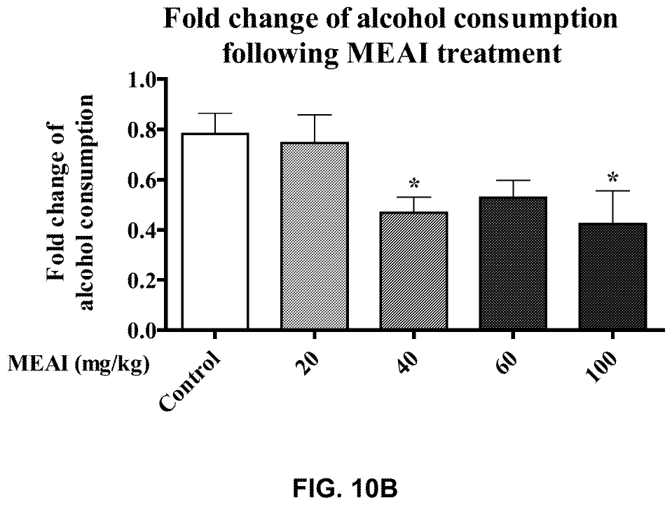

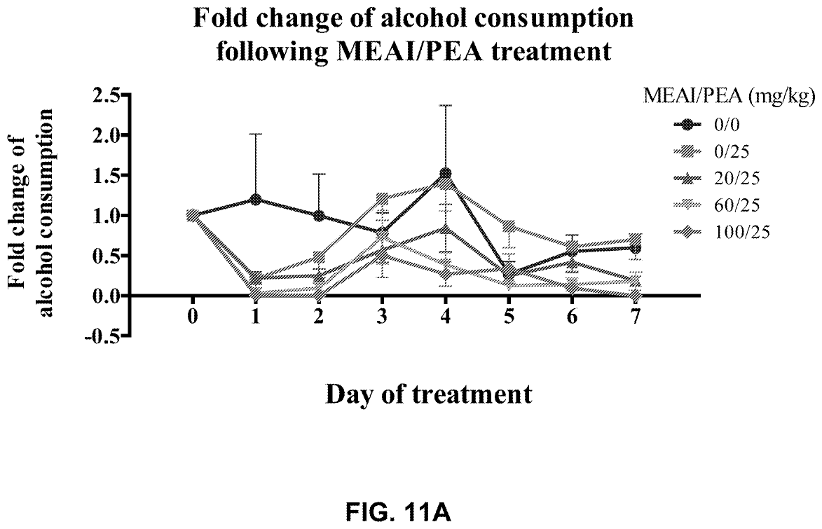

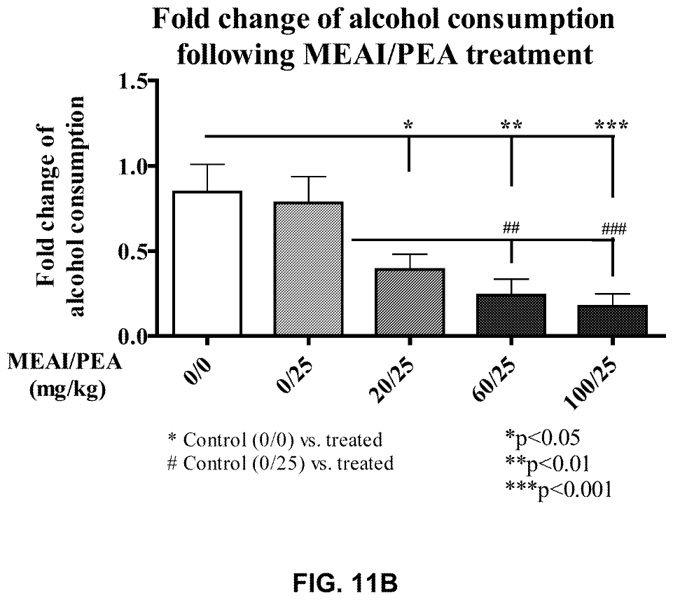

Intermittent access to 20% Alcohol in two-bottle choice (IA2BC) model in mice. The mice were subjected to 20% ethanol 24 hours, three times a week, for five weeks (15 alcohol consumption days), and the alcohol consumption was measured by weight. Following 5 weeks the mice were allocated to groups according to their alcohol consumption, and the effect of MEAI/PEA treatment in reducing alcohol consumption was examined. Mice were treated, with Vehicle/MEAI/PEA, daily, during days 36-48 in cycle 1, and during days 36-51 in cycle 2. FIGS. 3A and 3B demonstrate a significant reduction in alcohol consumption, during the 6/7 days of treatment, in the treated mice vs. control, in a dose dependent manner, in both cycles. Additional graphs summarizing the results of alcohol consumption are presented in FIGS. 7A and 7B, 8A and 8B, and 9A and 9B (Alcohol consumption during MEAI treatment) and FIGS. 10A and 10B, and 11A and 11B (Alcohol consumption Fold change). Statistical analysis was performed only on the 6/7 days of treatment, using two-way ANOVA, followed by Bonferroni post-hoc tests for multiple comparisons (*p<0.05; ** p<0.01; *** p<0.001).

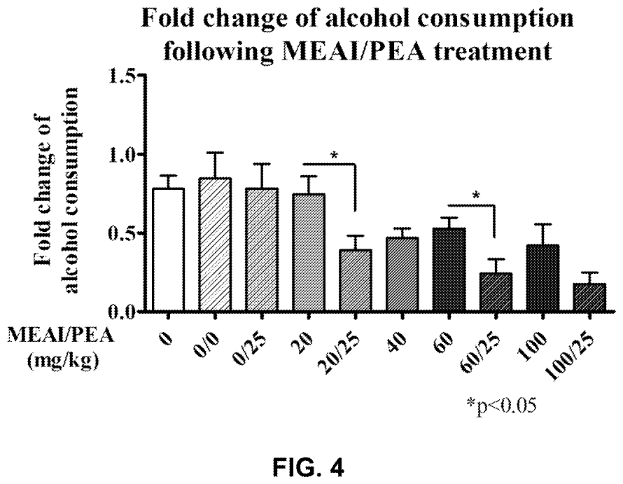

The averaged consumption before treatment was compared to the averaged consumption during treatment in each group. FIG. 4 represents the mean value of alcohol consumption of six alcohol days prior to MEAI treatment in each group (white bars), vs. mean value of six alcohol days of consumption during MEAI treatment in each group (blue bars). In cycle 1 the alcohol consumption was significantly reduced following treatment with MEAI at a dose of 40 mg/kg and at higher doses (** p<0.01; *** p<0.001) compared to consumption before treatment. In cycle 2 the alcohol consumption was significantly reduced following dual treatment with 25 mg/kg PEA in addition to MEAI at a dose of 20 mg/kg and at higher doses (** p<0.01, *** p<0.001) compared to consumption before treatment.

The averaged consumption before treatment was compared to the averaged consumption during treatment in each group. FIG. 3 represents the mean value of alcohol consumption of six alcohol days prior to MEAI treatment in each group (white bars), vs. mean value of six alcohol days of consumption during MEAI treatment in each group (blue bars). In cycle 1 (FIG. 3A) the alcohol consumption was significantly reduced following treatment with MEAI at a dose of 40 mg/kg and at higher doses (** p<0.01; *** p<0.001) compared to consumption before treatment. In cycle 2 (FIG. 3B) the alcohol consumption was significantly reduced following dual treatment with 25 mg/kg PEA in addition to MEAI at a dose of 20 mg/kg and at higher doses (** p<0.01, *** p<0.001) compared to consumption before treatment.

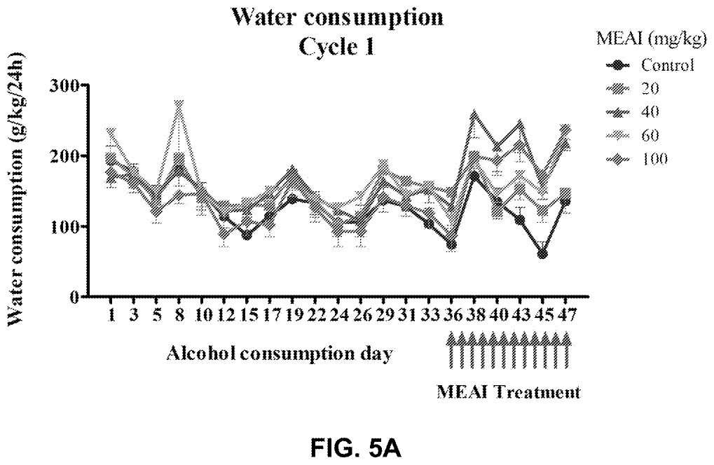

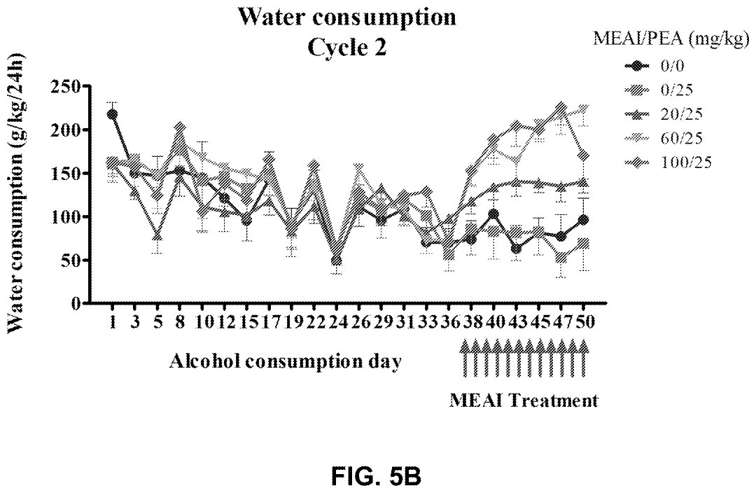

The additive effect of PEA was tested by comparing the consumption of alcohol in groups that were treated with the same dose of MEAI (20/60/100 mg/kg) with or without PEA (25 mg/kg). Fold change of each group consumption during 6-7 alcohol days with MEAI/PEA treatment out of its own baseline consumption prior to treatment were compared. Results in FIG. 5 show that in the lower doses of 20 and 60 mg/kg MEAI—there was a significant decrease in alcohol consumption in the combined treatment compared to mice treated with MEAI alone in the same dose without PEA (*p<0.05). At the highest dose of 100 mg/kg MEAI—there was a reduction in alcohol consumption following addition of PEA, however, it did not reach statistical significance.

The additive effect of PEA was tested by comparing the consumption of alcohol in groups that were treated with the same dose of MEAI (20/60/100 mg/kg) with or without PEA (25 mg/kg). Fold change of each group consumption during 6-7 alcohol days with MEAI/PEA treatment out of its own baseline consumption prior to treatment were compared. Results in FIG. 4 show that in the lower doses of 20 and 60 mg/kg MEAI—there was a significant decrease in alcohol consumption in the combined treatment compared to mice treated with MEAI alone in the same dose without PEA (*p<0.05). At the highest dose of 100 mg/kg MEAI—there was a reduction in alcohol consumption following addition of PEA, however, it did not reach statistical significance.

### Water Consumption:

Water consumption was monitored during alcohol treatment, three times a week, for five weeks. The effect of MEAI/PEA treatment on water consumption was examined. Mice were treated, with Vehicle/MEAI/PEA, daily, during days 36-48 in cycle 1, and during Days 36-51 in cycle 2. FIG. 5 shows that the consumption of water was elevated during the MEAI treatment, in both cycles. In cycle 2 (FIG. 5B) the water consumption was elevated at a dose dependent manner, and in reverse correlation with the alcohol consumption, which was reduced at a dose dependent manner.

### Alcohol Preference:

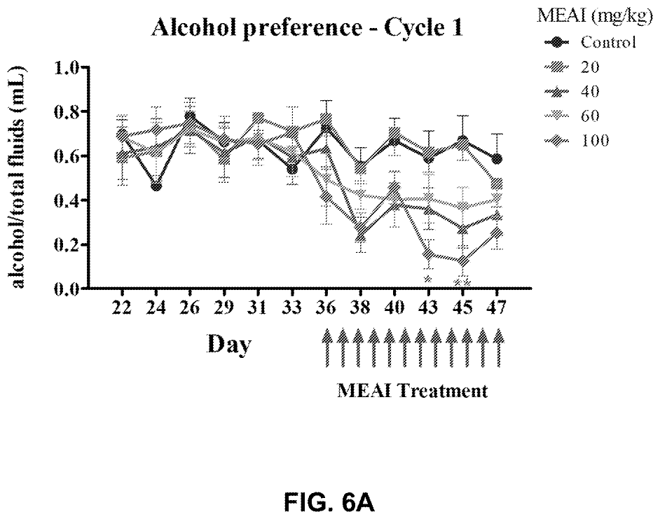

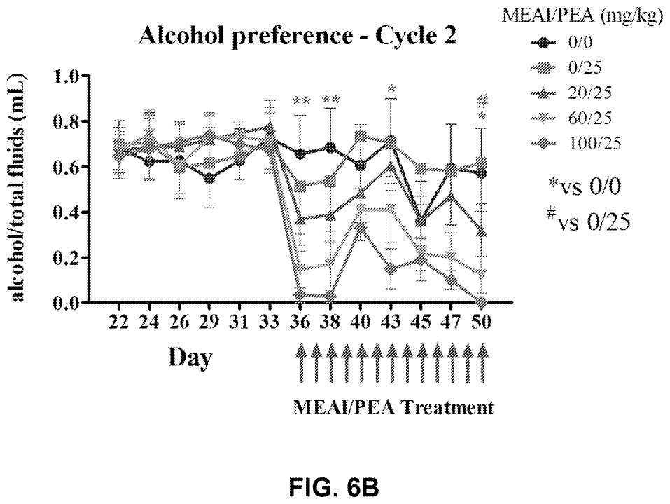

The mean alcohol preference, which was defined as the ratio between alcohol weight (g/kg/24 h) divided by the total weight of water and alcohol consumed by the animals (g/kg/24 h) was evaluated. As shown in FIGS. 6A and 6B, alcohol preference was reduced in a dose dependent manner following MEAI and MEAI/PEA treatment, compared to control groups. In both cycles, the reduction was statistically significant in the highest doses (100 mg/kg MEAI; 100/25 mg/kg MEAI/PEA), suggesting that the reduction in alcohol consumption is associated with elevation of water consumption.

### CONCLUSIONS

The efficiency of MEAI in reducing alcohol consumption in mice, at several MEAI doses and in combination with Palmitoylethanolamide (PEA) was evaluated in a model of intermittent access to 20% alcohol in two-bottle choice (IA2BC). The mice were provided with 20% alcohol solution, for 24 hours, three times a week, for 7 weeks, and were treated everyday with MEAI or MEAI/PEA during the last two weeks of alcohol treatment. The alcohol consumption was measured by weighing the alcohol buttle before and after; water consumption was measured in parallel. In addition, food consumption (during 24 hours) was measured before, during and after the MEAI/PEA treatment, during alcohol addition days.

Results showed a significant dose dependent effect for the MEAI treatment in reducing alcohol consumption of the treated animals, with additive effect for the PEA treatment at the lower MEAI doses. Furthermore, in reverse correlation, the consumption of water was elevated during the MEAI treatment, in both cycles, hence, the alcohol preference (out of total fluids) was also reduced, at a dose dependent manner.

There were no significant differences between the animals' body weight, and the food consumption was also not affected by the MEAI treatment.

During the study, mortality was observed in four animals from the high doses treated groups however, it is important to take into consideration that these animals were treated with MEAI following a prolonged time of alcohol consumption. Supporting evidence to the harmful effect of alcohol consumption can be found in the gross pathology analysis which exhibited macroscopic findings mostly in the liver, kidneys, heart, and spleen, of all groups including the control.

Based on the above findings and under this study conditions, MEAI exhibited a suppressive effect on mice alcohol consumption, in the IA2BC model, and the PEA improved this effect starting from the low MEAI doses.

The many features and advantages of the present disclosure are apparent from the detailed specification, and thus it is intended by the appended claims to cover all such features and advantages of the present disclosure that fall within the true spirit and scope of the present disclosure. Further, since numerous modifications and variations will readily occur to those skilled in the art, it is not desired to limit the present disclosure to the exact construction and operation illustrated and described and accordingly, all suitable modifications and equivalents may be resorted to, falling within the scope of the present disclosure.

Moreover, those skilled in the art will appreciate that the conception upon which this disclosure is based may readily be used as a basis for designing other structures, methods, and systems for carrying out the several purposes of the present disclosure. Accordingly, the claims are not to be considered as limited by the foregoing description or examples.

1. A pharmaceutical composition comprising 5-methoxy-2-aminoindan or a salt thereof, and an N-acylethanolamine or a salt thereof; and at least one pharmaceutically acceptable carrier and/or excipient.

2. The pharmaceutical composition of claim 1, wherein the pharmaceutical composition is a unit dosage form composition.

3. The pharmaceutical composition of claim 2, wherein the pharmaceutical composition is a solid unit dosage form composition or a liquid unit dosage form composition.

4. (canceled)

5. The pharmaceutical composition of claim 1, wherein the pharmaceutical composition is packaged as a single unit dose or as a plurality of single unit doses.

6. The pharmaceutical composition of claim 2, wherein the unit dosage form composition comprises from 30 mg to 130 mg of 5-methoxy-2-aminoindan.

7. The pharmaceutical composition of claim 1, wherein the pharmaceutical composition is formulated for oral administration.

8-17. (canceled)

18. The pharmaceutical composition of claim 1, wherein the 5-methoxy-2-aminoindan or salt thereof and the N-acylethanolamine or salt thereof is present in a molar ratio from about 1:0.2 to about 1:2000.

19. The pharmaceutical composition of claim 6, wherein the unit dosage form composition comprises about 70 mg of the 5-methoxy-2-aminoindan or salt thereof.

20. The pharmaceutical composition of claim 1, comprising:
a) about 200 mg to 1800 mg of the N-acylethanolamine or salt thereof;
b) about 250 mg to 1550 mg of the N-acylethanolamine or salt thereof;
c) about 300 mg to 1200 mg of the N-acylethanolamine or salt thereof;
d) about 350 mg to 950 mg of the N-acylethanolamine or salt thereof;
e) about 400 mg to 700 mg of the N-acylethanolamine or salt thereof; or
f) about 450 mg to 600 mg of the N-acylethanolamine or salt thereof.

21. The pharmaceutical composition of claim 1, comprising:
a) about 50 mg of the N-acylethanolamine or salt thereof;
b) about 100 mg of the N-acylethanolamine or salt thereof;
c) about 150 mg of the N-acylethanolamine or salt thereof;
d) about 200 mg of the N-acylethanolamine or salt thereof;
e) about 250 mg of the N-acylethanolamine or salt thereof;
f) about 400 mg of the N-acylethanolamine or salt thereof;
g) about 500 mg of the N-acylethanolamine or salt thereof;
h) about 750 mg of the N-acylethanolamine or salt thereof;
i) about 800 mg of the N-acylethanolamine or salt thereof;
j) about 1000 mg of the N-acylethanolamine or salt thereof; or
k) about 1500 mg of the N-acylethanolamine or salt thereof.

22. The pharmaceutical composition of claim 1, wherein the N-acylethanolamine or salt thereof is selected from N-palmitoylethanolamine (PEA), Me-palmitoylethanolamide (Me-PEA), palmitoylcyclohexamide, palmitoylbutylamide, oleoylethanolamine (OEA), palmitoylisopropylamide (PIA), and any combination thereof.

23. The pharmaceutical composition of claim 1, wherein the N-acylethanolamine or salt thereof comprises N-palmitoylethanolamine (PEA).

24. A method of regulating binge behavior, comprising administering to a subject in need thereof a therapeutically effective amount of 5-methoxy-2-aminoindan or a salt thereof, and a therapeutically effective amount of an N-acylethanolamine or a salt thereof.

25. The method of claim 24, wherein the binge behavior is associated with alcohol consumption, eating, tobacco consumption, shopping, or sexual conduct.

26. The method of claim 24, wherein the binge behavior is binge drinking.

27. The method of claim 24, wherein the therapeutically effective amount of the 5-methoxy-2-aminoindan or salt thereof comprises from 30 mg to 130 mg of 5-methoxy-2-aminoindan.

28. The method of claim 24, wherein the therapeutically effective amount of the N-acylethanolamine or salt thereof comprises:
a) about 200 mg to 1800 mg of the N-acylethanolamine or salt thereof;
b) about 250 mg to 1550 mg of the N-acylethanolamine or salt thereof;
c) about 300 mg to 1200 mg of the N-acylethanolamine or salt thereof;
d) about 350 mg to 950 mg of the N-acylethanolamine or salt thereof;
e) about 400 mg to 700 mg of the N-acylethanolamine or salt thereof; or
f) about 450 mg to 600 mg of the N-acylethanolamine or salt thereof.

29. The method of claim 24, wherein the therapeutically effective amount of the N-acylethanolamine or salt thereof comprises:
a) about 50 mg of the N-acylethanolamine or salt thereof;
b) about 100 mg of the N-acylethanolamine or salt thereof;
c) about 150 mg of the N-acylethanolamine or salt thereof;
d) about 200 mg of the N-acylethanolamine or salt thereof;
e) about 250 mg of the N-acylethanolamine or salt thereof;
f) about 400 mg of the N-acylethanolamine or salt thereof;
g) about 500 mg of the N-acylethanolamine or salt thereof;
h) about 750 mg of the N-acylethanolamine or salt thereof;
i) about 800 mg of the N-acylethanolamine or salt thereof;
j) about 1000 mg of the N-acylethanolamine or salt thereof; or
k) about 1500 mg of the N-acylethanolamine or salt thereof.

30. The method of claim 24, wherein the N-acylethanolamine or salt thereof is selected from N-palmitoylethanolamine (PEA), Me-palmitoylethanolamide (Me-PEA), palmitoylcyclohexamide, palmitoylbutylamide, oleoylethanolamine (OEA), palmitoylisopropylamide (PIA), and any combination thereof.

31. The method of claim 24, wherein the N-acylethanolamine or salt thereof comprises N-palmitoylethanolamine (PEA).

32. The method of claim 24, wherein the therapeutically effective amount of the 5-methoxy-2-aminoindan or salt thereof and the therapeutically effective amount of the N-acylethanolamine or salt thereof are formulated in the pharmaceutical composition of claim 1.

33. A method of regulating alcohol consumption, comprising administering to a subject in need thereof the pharmaceutical composition of claim 1.

34. A method of increasing a preference for consuming water compared to consuming alcohol, comprising administering to a subject in need thereof the pharmaceutical composition of claim 1.

35. A method of increasing water consumption in a subject, comprising administering to a subject in need thereof a therapeutically effective amount of 5-methoxy-2-aminoindan or a salt thereof.

36. A method of increasing water consumption in a subject, comprising administering to a subject in need thereof the pharmaceutical composition of claim 1.

37. A method of increasing a preference for consuming water compared to consuming alcohol, comprising administering to a subject in need thereof a therapeutically effective amount of 5-methoxy-2-aminoindan or a salt thereof.
